(19) United States
(12) Patent Application Publication
(10) Pub. No.: US 2026/0242458 A1
(43) Pub. Date: Aug. 20, 2026

(54) ANTI-IL-25 ANTIBODIES AND METHODS OF USE THEREOF

(71) Applicant: NOVAROCK BIOTHERAPEUTICS, LTD., Ewing, NJ (US)

(72) Inventors: Haichun HUANG, Yardley, PA (US); Ming LEI, Princeton, NJ (US); Yi PEI, Paoli, PA (US); Han LI, Newtown, PA (US); Yick Loi Raymond YU, East Brunswick, NJ (US); Zhiqiang ZHU, West Windsor, NJ (US)

(21) Appl. No.: 19/469,782

(22) PCT Filed: Mar. 29, 2024
(86) PCT No.: PCT/US2024/022156

§ 371 Date: Sep. 26, 2025

Related U.S. Application Data
(60) Provisional application No. 63/493,453, filed on Mar. 31, 2023.

(51) Int. Cl. C07K 16/24; A61P 11/06

(52) U.S. Cl. CPC C07K 16/244; A61P 11/06; C07K 2317/20; C07K 2317/21; C07K 2317/24; C07K 2317/31

(57) ABSTRACT

The present disclosure provides antibodies and antibody fragments thereof that bind to IL-25. The disclosed antibodies and antibody fragments thereof can modulate a biological activity of the IL-25/IL-25 receptor signaling axis and are therefore useful for the treatment of Type 2 inflammation diseases, autoimmune diseases, allergic disorders or cancer.

### FIELD

The present disclosure generally relates to antibodies and antibody fragments thereof that bind interleukin-25. The antibodies may be useful for the prevention and/or treatment of Type 2 inflammation diseases, autoimmune diseases, or cancer.

### BACKGROUND

The interleukin-17 (IL-17) family belongs to a group of cytokines that play a crucial role in inflammatory responses during autoimmune pathogenesis and host defense against extracellular pathogens. IL-25 (also known as IL-17E) is produced not only by immune cells (e.g., T cells, dendritic cells, and macrophages) but also other non-immune cells (e.g., fibroblasts, epithelial cells, and keratinocytes) and even some cancer cells (such as melanoma, liver, breast, and cervical cancers) (Gowhari Shabgah, A., et al., Cancer Med. (2021) 10:5191-5202). The diversity of cellular sources suggests that IL-25 participates in many immune responses and cellular processes.

IL-25 is distinctly different from other IL-17 family members in terms of both molecular structure and its biological function. As an epithelial cell cytokine, IL-25 along with TSLP and IL-33 is tasked with the role of an “alarmin” or barrier surface cytokine and alerts the immune system about extrinsic environmental threats (such as allergens, bacteria, viruses, and helminths) and mobilizes host immune defense mechanisms (Ham, J. et al., Immune Netw. (2022) (1):e11, Borowczyk J. et al, Journal of Allergy and Clinical Immunology (2021) 148(1) 40-52). Recent studies suggest that epithelial cytokines also have a broad range of effector functions in numerous pathological conditions, including Type 2 inflammatory diseases, allergic disorders, viral infections, chronic inflammatory disorders, auto-immune conditions, and cancer.

IL-25 plays a dual role in regulating immune responses and autoimmune disease pathogenesis. As a pro-inflammatory cytokine, IL-25 exacerbates allergic inflammation by promoting the production of Th2 cytokines including IL-4, IL-5 and IL-13 by Th2 cells. Moreover, IL-25 can induce the proliferation and activation of innate immune cells, the production of other pro-inflammatory cytokines and the recruitment of immune cells (Deng, C. et al., Front. Immunol (2021) 12:691559).

On the other hand, IL-25 has been reported to play a role in autoimmune diseases as an anti-inflammatory cytokine and an inhibitor of both innate and adaptive immunity (Saadoun, D. et al., Current pharmaceutical design (2011) 17:3781-3785. For example, the anti-inflammatory and immunosuppressive activities of IL-25 in rheumatoid arthritis are attributed to an IL-13-dependent downregulation of the Th17 cell response (Liu, D. et al., Sci Rep (2016) 6:36002).

Additionally, the expression of IL-25 and its receptor have been dysregulated in various cancers compared with normal tissues. Therefore, it has been indicated that IL-25 might also play a Janus-faced role in cancer progression or regression. The tumor-suppressive role of IL-25 is mainly attributed to the infiltration of eosinophils and B cells into the tumor microenvironment and induction of apoptosis. In contrast, its tumor supportive roles rely on the deviation of immune responses and stimulation of EMT and cell growth (Gowhari Shabgah, A. et al., Cancer Med. (2021) 10: 5191-5202).

Although the IL-25 pathway is a recognized therapeutic target, only a single Phase I clinical trial evaluating the safety, tolerability and pharmacokinetic characteristics IL-25 has been identified to date. Accordingly, there is an unmet need in the art for novel IL-25 antagonists, such as the anti-IL-25 antibodies disclosed herein, for the treatment of diseases or disorders associated with IL-25 expression and/or signaling.

### SUMMARY

The present disclosure addresses the above need by providing antibodies and antibody fragments thereof that bind to IL-25 including human IL-25. The antibodies and antibody fragments thereof are useful for the treatment of immune-mediated inflammatory diseases (IMIDs) (e.g., autoimmune diseases and inflammatory disorders) and cancer. The anti-IL-25 antibodies or antibody fragments thereof can either be used alone (e.g., as a monotherapy) or in combination with other immunotherapeutic agents. In an alternative embodiment, an antibody fragment of an IL-25 antibody disclosed herein can be used as a component of a bispecific or multi-specific antibody or fusion protein.

In some embodiments, the anti-IL-25 antibody or antibody fragment thereof binds to the IL-25 cytokine (IL-17E) with high affinity, and does not bind (e.g., specifically bind) to any other members of the IL-17 cytokine family.

In some embodiments, the anti-IL-25 antibody or antibody fragment thereof comprises a heavy chain variable region comprising CDR1: SEQ ID NO: 11, CDR2: SEQ ID NO: 12, and CDR3: SEQ ID NO: 13; and/or a light chain variable region comprising CDR1: SEQ ID NO: 14. CDR2: SEQ ID NO: 15, and CDR3: SEQ ID NO: 16.

In some embodiments, the anti-IL-25 antibody or antibody fragment thereof comprises a heavy chain variable region comprising CDR1: SEQ ID NO: 17, CDR2: SEQ ID NO: 18, and CDR3: SEQ ID NO: 19; and/or a light chain variable region comprising CDR1 SEQ ID NO: 20, CDR2: SEQ ID NO: 21, and CDR3 SEQ ID NO 22.

In some embodiments, the anti-IL-25 antibody or antibody fragment thereof comprises a heavy chain variable region comprising CDR1: SEQ ID NO: 23, CDR2: SEQ ID NO: 24, and CDR3: SEQ ID NO: 25; and/or a light chain variable region comprising CDR1: SEQ ID NO: 26, CDR2: SEQ ID NO: 27, and CDR3: SEQ ID NO: 28.

In some embodiments, the anti-IL-25 antibody or antibody fragment thereof comprises a heavy chain variable region comprising CDR1: SEQ ID NO: 29, CDR2: SEQ ID NO: 30, and CDR3: SEQ ID NO: 31; and/or a light chain variable region comprising CDR1: SEQ ID NO: 32, CDR2: SEQ ID NO: 33, and CDR3: SEQ ID NO: 34.

In some embodiments, the anti-IL-25 antibody or antibody fragment thereof comprises a heavy chain variable region comprising CDR1: SEQ ID NO: 35, CDR2: SEQ ID NO: 36, and CDR3: SEQ ID NO: 37; and/or a light chain variable region comprising CDR1: SEQ ID NO: 38. CDR2: SEQ ID NO: 39, and CDR3: SEQ ID NO: 40.

In some embodiments, the anti-IL-25 antibody or antibody fragment thereof comprises a heavy chain variable region comprising CDR1: SEQ ID NO:23 CDR2: SEQ ID NO: 49, and CDR3: SEQ ID NO: 25; and/or a light chain variable region comprising CDR1. SEQ ID NO: 50 CDR2. SEQ ID NO:51, and CDR3: SEQ ID NO: 28.

In some embodiments, the anti-IL-25 antibody or antibody fragment thereof comprises a heavy chain variable region comprising CDR1: SEQ ID NO:23 CDR2: SEQ ID NO 52 and CDR3 SEQ ID NO: 25; and/or a light chain variable region comprising CDR1: SEQ TD NO: 50. CDR2: SEQ ID NO: 51, and CDR-3: SEQ ID NO: 28.

In some embodiments, the anti-IL-25 antibody or antibody fragment thereof comprises a heavy chain variable region having an amino acid sequence selected from the group consisting of SEQ ID NOs: 1, 3, 5, 7, and 9.

In other embodiments, the anti-IL-25 antibody or antibody fragment thereof comprises a light chain variable region having an amino acid sequence selected from the group consisting of SEQ ID NOs: 2, 4, 6, 8, and 10.

In other embodiments, the anti-IL-25 antibody or antibody fragment thereof comprises a heavy chain variable region having an amino acid sequence selected from the group consisting of SEQ ID NOs: 1, 3, 5, 7, and 9 and a variable light chain sequence selected from the group consisting of SEQ ID NOs: 2, 4, 6, 8 and 10.

In some embodiments, the anti-IL-25 antibody or antibody fragment thereof comprises a humanized heavy chain variable region having an amino acid sequence selected from the group consisting of SEQ ID NOs: 44 and 47.

In other embodiments, the anti-IL-25 antibody or antibody fragment thereof comprises a humanized light chain variable region having an amino acid sequence selected from the group consisting of SEQ ID NOs: 45, 46 and 48.

In other embodiments, the anti-IL-25 antibody or antibody fragment thereof comprises a heavy chain variable region having an amino acid sequence selected from the group consisting of SEQ ID NOs: 44 and 47 and a variable light chain sequence selected from the group consisting of SEQ ID NOs: 45, 46 and 48.

In some embodiments, the anti-LL-25 antibody or antibody fragment thereof comprises a heavy chain variable region sequence and a light chain variable region sequence, selected from the following combinations:
(a) a variable heavy chain sequence comprising SEQ ID NO: 1 and a variable light chain sequence comprising SEQ ID NO: 2;
(b) a variable heavy chain sequence comprising SEQ ID NO: 3 and a variable light chain sequence comprising SEQ ID NO: 4;
(c) a variable heavy chain sequence comprising SEQ ID NO: 5 and a variable light chain sequence comprising SEQ ID NO: 6;
(d) a variable heavy chain sequence comprising SEQ ID NO: 7 and a variable light chain sequence comprising SEQ ID NO: 8;
(e) a variable heavy chain sequence comprising SEQ ID NO: 9 and a variable light chain sequence comprising SEQ ID NO: 10;
(f) a variable heavy chain sequence comprising SEQ ID NO: 44 and a variable light chain sequence comprising SEQ ID NO: 45;
(g) a variable heavy chain sequence comprising SEQ ID NO: 41 and a variable light chain sequence comprising SEQ ID NO: 46 or
(h) a variable heavy chain sequence comprising SEQ ID NO: 47 and a variable light chain sequence comprising SEQ ID NO: 48.

In some embodiments, the antibody is an human anti-IL-25 antibody.

In some embodiments, the antibody is a full-length antibody.

In some embodiments, the antibody is a full-length antibody comprising a human IgG1 constant region selected from SEQ ID NO: 55 or SEQ ID NO: 56.

In some embodiments, the antibody fragment is selected from the group consisting of Fab, Fab, F(ab)2, Fd, Fv, scFv and scFv-Fc fragment, a single-chain antibody, a minibody, and a diabody.

In some embodiments, the antibody is a monoclonal antibody.

In some embodiments, the antibody is a human antibody (e.g, a fully human antibody).

In some embodiments, the antibody is a murine antibody.

In some embodiments, the antibody is a chimeric antibody.

In some embodiments, the antibody is a bispecific antibody.

In some embodiments, the antibody is a humanized antibody.

The present disclosure also provides a pharmaceutical composition comprising the antibody or antibody fragment thereof disclosed herein and a pharmaceutically acceptable carrier.

Further provided by the present disclosure are methods of treating and/or preventing a Type 2 inflammation disease, autoimmune disease, allergic disorder, or cancer in a subject in need thereof, the method comprising: administering to the subject the antibody or antibody binding fragment as disclosed herein.

The present disclosure also provides a composition of polynucleotides comprising a first polynucleotide coding for a heavy chain variable region comprising the amino acid sequence set forth in SEQ ID NOs: 1, 3, 5, 7, 9, 44 or 47; and a second polynucleotide coding for a light chain variable region comprising the amino acid sequence set forth in SEQ ID NOs: 2, 4, 6, 8, 10, 45, 46 or 48.

Additionally, the present disclosure provides a composition of vectors comprising a first vector comprising a first polynucleotide as disclosed herein (e.g., a polynucleotide coding for SEQ ID NOs: SEQ ID NO: 1, 3, 5, 7, 9, 44 or 47; and a second vector comprising a second polynucleotide as disclosed herein (e.g., a polynucleotide coding for SEQ ID NOs: 2, 4, 6, 8, 10, 45, 46 or 48.

The present disclosure also provides a cell (e.g., a CHO cell) comprising a polynucleotide composition disclosed herein, or a vector composition disclosed herein.

Also provided by the present disclosure are methods for the production of an anti-IL-25 antibody or antibody fragment thereof as disclosed herein that comprise culturing a cell disclosed herein that expresses an IL-25 antibody or antibody fragment thereof in a culture media; and recovering the anti-IL-25 antibody or the antibody fragment thereof from the media.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIGS. 1A-1D provide the amino acid sequences of the VH and VL domains of anti-IL-25 antibodies and their respective CDR sequences. Sequence identifiers are provided and the CDRs as determined by Kabat are underlined in the context of the variable domain sequence.

FIGS. 2A-B provides the amino acid sequence of human, mouse and cynomolgus monkey IL-25, and the amino acid sequences of the human or mouse antibody constant regions.

FIGS. 3A-3C show the binding of anti-IL-25 antibodies to recombinant human, mouse and cynomolgus IL-25 proteins. ELISA utilizing goat-anti-human IgG-HRP as detection demonstrates the binding of anti-IL-25 antibodies to recombinant human (3A) and mouse (3B) IL-25 proteins. ELISA utilizing goat-anti-human Kappa Light Chain antibody HRP as detection demonstrates the binding of anti-IL-25 antibodies to cynomolgus (3C) IL-25 proteins.

FIG. 4 demonstrates that anti-IL-25 antibodies inhibit NFkB signaling induced by human IL-25 in HEK-Blue IL-17 reporter cells.

FIG. 5 shows that anti-IL-25 antibodies inhibit IL-25 induced CXCL-1 production in human colon cancer cell line HT29.

FIG. 6 shows that anti-IL-25 antibodies inhibit IL-25 induced IL-5 production in a human PBMC assay.

FIGS. 7A-7B show that anti-IL-25 antibodies reduce airway resistance in the OVA-induced asthma model. The airway hyperresponsiveness (AHR) of OVA-sensitized/challenged BALB/c mice to methacholine challenge. The AHR of each mouse is measured using Buxo's Whole Body Plethysmography (WBP) system on Day 31. AHR is expressed as percentage change from the baseline level of lung resistance (Penh value). The dose-response data are presented as group means f SEM (7A) and the AUC of % of baseline Penh (7B). ** p<0.01, *** p<0.001 compared to OVA+Vehicle using repeat measurement/Bonferroni's (7A) and ** p<0.01, *** p<0.001 compared to OVA+Vehicle using one-way Anova/Dunnett's (7B).

FIG. 8 demonstrates that anti-IL-25 antibodies inhibit the IL-5 production in lung of OVA-induced asthma model. ** p<0.01, *** p<0.001 compared to OVA+Vehicle using one-way Anova/Dunnett's.

FIGS. 9A-9C show the binding of humanized anti-IL-25 antibodies to recombinant human IL-25 (9A), cynomolgus IL-25 (9B) and mouse IL-25 (9C) measured by ELISA.

FIG. 10 demonstrates that humanized anti-IL-25 antibodies inhibit NFkB signaling induced by human IL-25 in HEK-Blue IL-17 reporter cells.

FIG. 11 shows that humanized anti-IL-25 antibodies inhibit IL-25 induced CXCL-1 production in human colon cancer cell line HT29.

FIG. 12 shows that humanized anti-IL-25 antibodies inhibit IL-25 induced IL-5 production in a human PBMC assay.

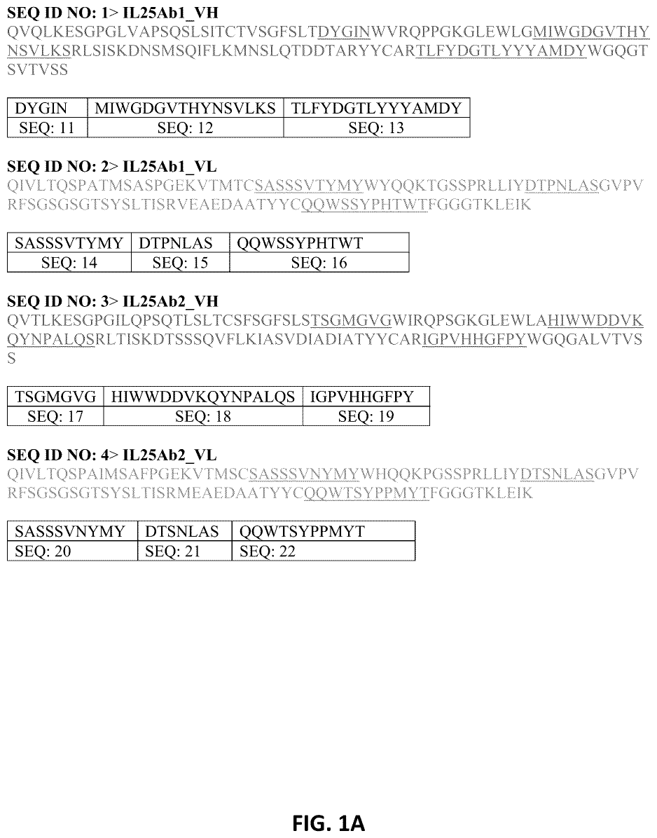

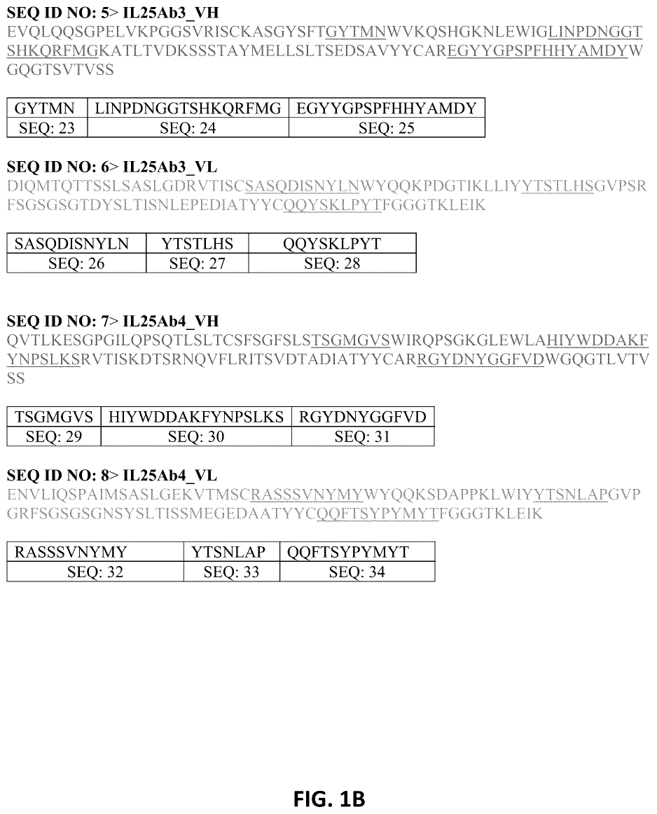

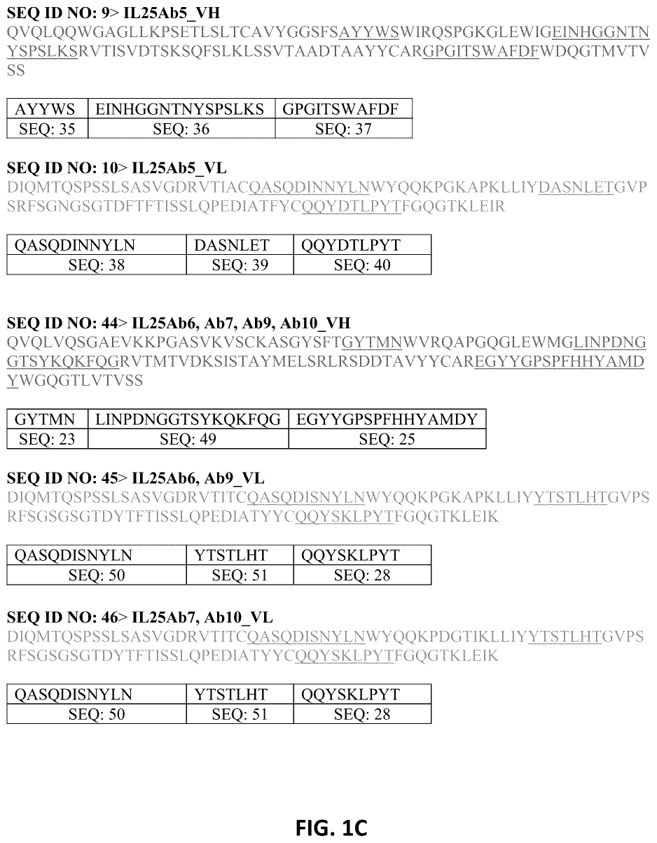

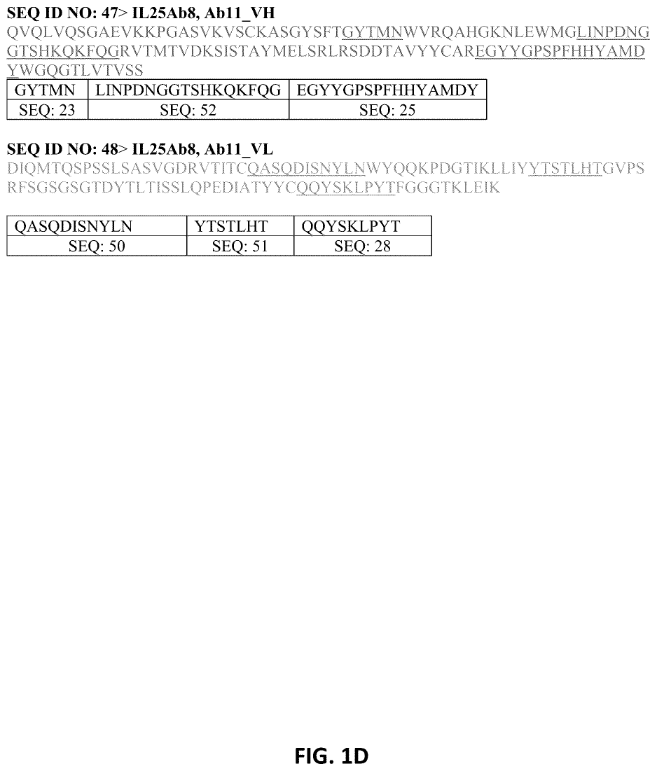

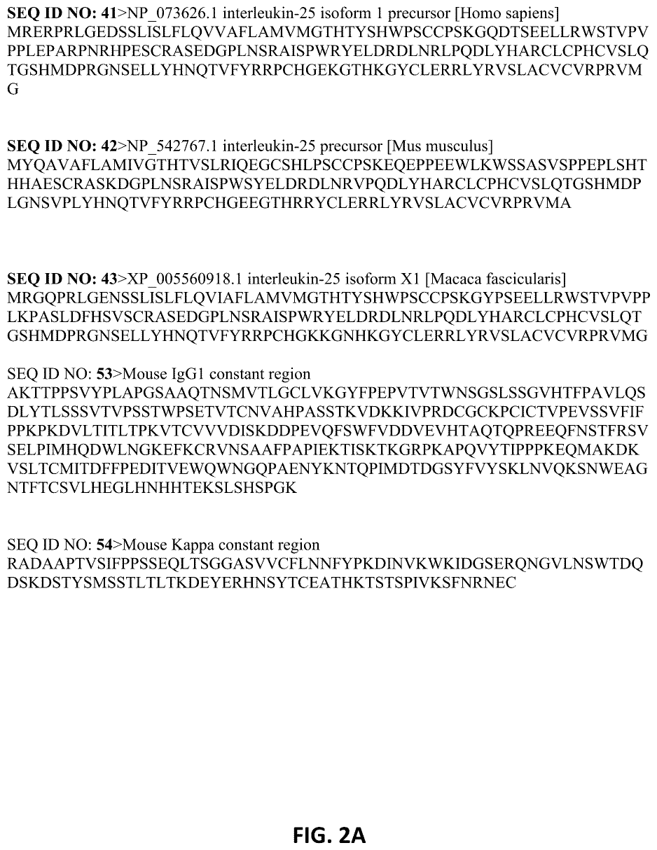

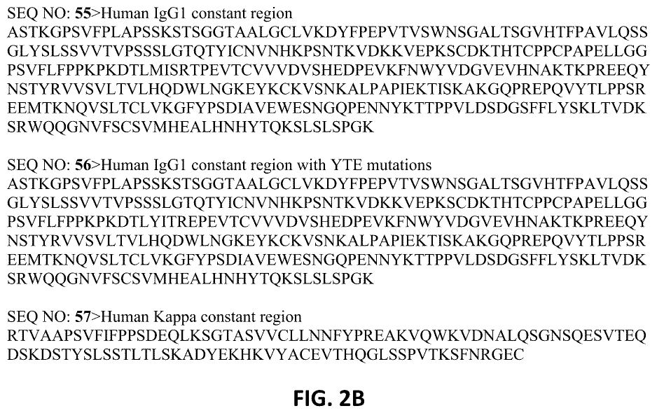

### DETAILED DESCRIPTION

So that the disclosure may be more readily understood, certain technical and scientific terms are specifically defined below. Unless specifically defined elsewhere in this document, all other technical and scientific terms used herein have the meaning commonly understood by one of ordinary skill in the art to which this disclosure belongs.

Throughout this disclosure the following abbreviations will be used:
mAb or Mab or MAb—Monoclonal antibody.
CDR—Complementarity determining region in the immunoglobulin variable regions.
HCDR—Heavy chain complementarity-determining region.
LCDR—Light chain complementarity-determining regions.
VH—Immunoglobulin heavy chain variable region.
VL—Immunoglobulin light chain variable region
FR—Antibody framework region, the immunoglobulin variable regions excluding the CDR regions.

As used herein the term “interleukin-25” (used interchangeably with “IL-25”) refers to the native-sequence polypeptide, isoforms, chimeric polypeptides, all homologs, fragments, and precursors of human IL-25. The amino acid sequences for human, cynomolgus, and murine IL-25 are provided in NCBI Reference Sequences: NP_073626.1 (human) (SEQ ID NO: 41), XP_005560919.1 (cynomolgus monkey) (SEQ ID NO: 43), and NP_542767.1 (mouse) (SEQ ID NO. 42). Orthologs of IL-25 share ~92% and ~80% homology to the human protein in cynomolgus monkey and mice, respectively.

As used herein the term “IL-25 complex” refers to IL-17RA/IL-17RB complex, also known as IL-25 receptor.

As used herein the term “IL-17RA” refers to Interleukin 17 receptor A, also known as CDw217 (cluster of differentiation w217). The protein encoded by this gene (interleukin 17A receptor; IL-17RA) is a ubiquitous type 1 membrane glycoprotein that binds with low affinity to interleukin 17A.

As used herein the term “IL-17RB” refers to Interleukin-17 receptor B. This receptor specifically binds to IL-17B and IL-17E (IL-25) but does not bind to either IL-17 A or IL-17C.

The term “antibody” herein is used in the broadest sense and encompasses various antibody structures, including but not limited to monoclonal antibodies, polyclonal antibodies, and multi-specific antibodies (e.g., bispecific antibodies).

An exemplary antibody such as an IgG comprises two heavy chains and two light chains. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino terminus to carboxy-terminus in the following order: FR1, CDR1, FR2, CDR2, FR3, CDR3, FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917.

The term “monoclonal antibody” as used herein refers to an antibody obtained from a population of substantially homogeneous antibodies, e.g., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to a recombinant antibody in which a portion of the heavy and/or light chain is derived from a particular source or species, while the remainder of the heavy and/or light chain is derived from a different source or species.

A “human antibody” is an antibody that possesses an amino-acid sequence corresponding to that of an antibody produced by a human and/or has been made using any of the techniques for making human antibodies known to one of skill in the art. This definition of a human antibody specifically excludes a humanized antibody comprising non-human antigen-binding residues. Human antibodies can be produced using various techniques known in the art, including methods described in Cole et al, Monoclonal Antibodies and Cancer Therapy, Alan R. Liss, p. 77 (1985); Boerner et al, J. Immunol, 147(I):86-95 (1991). See also van Dijk and van de Winkel, Curr. Opin. Pharmacol, 5: 368-74 (2001). Human antibodies can be prepared by administering the antigen to a transgenic humanized animal that has been modified to produce such antibodies in response to antigenic challenge, but whose endogenous loci have been disabled, e.g., immunized HuMab mice (see, e.g., Nils Lonberg et al., 1994, Nature 368:856-859, WO 98/24884, WO 94/25585, WO 93/1227, WO 92/22645, WO 92/03918 and WO 01/09187 regarding HuMab mice), xenomice (see, e.g., U.S. Pat. Nos. 6,075,181 and 6,150,584 regarding XENOMOUSE™ technology) or Trianni mice (see, e.g., WO 2013/063391, WO 2017/035252 and WO 2017/136734).

The term “humanized antibody” refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those skilled in the art will be aware of humanized antibodies and will also be aware of suitable techniques for their generation. See for example, Hwang, W. Y. K., et al., Methods 36:35, 2005: Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989, U.S. Pat. Nos. 5,225,539, 5,530,101, 5,585,089; 5,693,761; 5,693,762; 6,180,370; and Selick et al, WO 90/07861, each of which is incorporated herein by reference in its entirety.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The terms “antigen-binding domain” of an antibody (or simply “binding domain”) of an antibody or similar terms refer to one or more fragments of an antibody that retain the ability to specifically bind to an antigen complex. Examples of binding fragments encompassed within the term “antigen-binding portion” of an antibody include (i) Fab fragments, monovalent fragments consisting of the VL, VH, CL and CH domains; (ii) F(ab′)2 fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341: 544-546), which consist of a VH domain; (vi) isolated complementarity determining regions (CDR), and (vii) combinations of two or more isolated CDRs which may optionally be joined by a synthetic linker.

“Complementarity determining region” or “CDR” as the terms are used herein refer to short polypeptide sequences within the variable region of both heavy and light chain polypeptides that are primarily responsible for mediating specific antigen recognition. There are three CDRs (termed CDR1, CDR2, and CDR3) within each VL and each VH.

As will be appreciated by those in the art, the exact numbering and placement of the CDRs can be different among different numbering systems. However, it should be understood that the disclosure of a variable heavy and/or variable light sequence includes the disclosure of the associated CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3).

In certain embodiments, the CDRs of an antibody can be determined according to the IMGT numbering system as described in Lefranc M-P, (1999) The Immunologist 7: 132-136 and Lefranc M-P et al, (1999) Nucleic Acids Res 27: 209-212, each of which is herein incorporated by reference in its entirety. Unless stated otherwise herein, references to residue numbers in the variable domain of antibodies means residue numbering by the Kabat numbering system.

In other embodiments, the CDRs of an antibody can be determined according to MacCallum R M et al, (1996) J Mol Biol 262: 732-745, herein incorporated by reference in its entirety. See also, e.g., Martin A. “Protein Sequence and Structure Analysis of Antibody Variable Domains,” in Antibody Engineering, Kontermann and Diibel, eds., Chapter 31, pp. 422-439, Springer-Verlag, Berlin (2001), herein incorporated by reference in its entirety. In other embodiments, the CDRs of an antibody can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.), herein incorporated by reference in its entirety.

“Framework” or “framework region” or “FR” refers to variable domain residues other than hypervariable region (HVR) residues. The FR of a variable domain generally consists of four FR domains: FR1, FR2, FR3, and FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In one embodiment, for the VL, the subgroup is subgroup kappa I as in Kabat et al., supra. In one embodiment, for the VH, the subgroup is subgroup III as in Kabat et al., supra.

The “hinge region” is generally defined as stretching from 216-238 (EU numbering) or 226-251 (Kabat numbering) of human IgG1. The hinge can be further divided into three distinct regions, the upper, middle (e.g., core), and lower hinge.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md. (1991).

A “blocking” antibody or an “antagonist” antibody is one which inhibits or reduces biological activity of the antigen it binds. Certain blocking antibodies or antagonist antibodies substantially or completely inhibit the biological activity of the antigen.

An “antibody that binds to the same epitope” as a reference antibody refers to an antibody that contacts an overlapping set of amino acid residues of the antigen as compared to the reference antibody or blocks binding of the reference antibody to its antigen in a competition assay by 50% or more. The amino acid residues of an antibody that contact an antigen can be determined, for example, by determining the crystal structure of the antibody in complex with the antigen or by performing hydrogen/deuterium exchange. In some embodiments, residues of an antibody that are within 5 Å the antigen are considered to contact the antigen. In some embodiments, an antibody that binds to the same epitope as a reference antibody blocks binding of the reference antibody to its antigen in a competition assay by 50% or more, and conversely, the reference antibody blocks binding of the antibody to its antigen in a competition assay by 50% or more.

The term “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab)2; diabodies; linear antibodies; single-chain antibody molecules (e.g., scFv). Papain digestion of antibodies produces two identical antigen-binding fragments, called “Fab” fragments, and a residual “Fc” fragment, a designation reflecting the ability to crystallize readily. The Fab fragment consists of an entire light (L) chain along with the variable region domain of the heavy (H) chain (VH), and the first constant domain of one heavy chain (CH1). Pepsin treatment of an antibody yields a single large F(ab)2 fragment which roughly corresponds to two disulfide linked Fab fragments having divalent antigen-binding activity and is still capable of cross-linking antigen. Fab fragments differ from Fab′ fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) approaches. For a review of methods for assessment of antibody purity, see, for example, Flatman et al., J. Chromatogr. B 848:79-87 (2007). In an embodiment, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain.

With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹M or lower, alternatively 10⁻¹⁰ M or lower, alternatively 10⁻¹¹ M or lower, alternatively 10⁻¹² M or lower or a Kd in the range of 10⁻⁴ M to 10⁻⁶ M or 10⁻⁶ M to 10⁻¹⁰ M or 10⁻⁷ M to 10⁻⁹M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope. As used herein the terms “specific binding,” “specifically binds,” and “selectively binds,” refer to antibody binding to an epitope of interleukin-25.

The term “affinity,” as used herein, means the strength of the binding of an antibody to an epitope. The affinity of an antibody is given by the dissociation constant Kd, defined as [Ab]-[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by V/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92-589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

An “epitope” is a term of art that indicates the site or sites of interaction between an antibody and its antigen(s). As described by (Janeway, C. Jr., P. Travers, et al. (2001). Immunobiology: the immune system in health and disease. Part II, Section 3-8. New York, Garland Publishing, Inc.): “An antibody generally recognizes only a small region on the surface of a large molecule such as a protein. [Certain epitopes] are likely to be composed of amino acids from different parts of the [antigen] polypeptide chain that have been brought together by protein folding. Antigenic determinants of this kind are known as conformational or discontinuous epitopes because the structure recognized is composed of segments of the protein that are discontinuous in the amino acid sequence of the antigen but are brought together in the three-dimensional structure. In contrast, an epitope composed of a single segment of polypeptide chain is termed a continuous or linear epitope” (Janeway, C. Jr., P. Travers, et al. (2001). Immunobiology: the immune system in health and disease. Part II, Section 3-8. New York, Garland Publishing, Inc.).

The term “KD”, as used herein, is intended to refer to the dissociation constant of a particular antibody-antigen interaction. It is calculated by the formula: Koff/Kon=KD.

The term “IC₅₀”, as used herein, is intended to refer to the effective concentration of antibody of the present invention needed to inhibit a specific biological or biochemical function by 50%.

“EC₅₀” with respect to an agent and a particular activity (e.g., binding to a cell, inhibition of enzymatic activity, activation or inhibition of an immune cell), refers to the efficient concentration of the agent which produces 50% of its maximum response or effect with respect to such activity. “EC₁₀₀)” with respect to an agent and a particular activity refers to the efficient concentration of the agent which produces its substantially maximum response with respect to such activity.

As used herein the term “antibody-based immunotherapy” and “immunotherapy” are used to broadly refer to any form of therapy that relies on the targeting specificity of an anti-IL-25 antibody, bispecific molecule, multi-specific molecule, binding agent, or fusion protein comprising an IL-25 specific binding agent, to mediate a direct or indirect effect on a cell characterized by aberrant expression of IL-25. The terms are meant to encompass methods of treatment using naked antibodies, bispecific antibodies (including T-cell engaging, NK cell engaging and other immune cell/effector cell engaging formats), antibody drug conjugates, cellular therapies using T-cells (CAR-T) or NK cells (CAR-NK) engineered to comprise an IL-25-specific chimeric antigen receptor, and oncolytic viruses comprising an IL-25 specific binding agent, and gene therapies by delivering the antigen binding sequences of the anti-IL-25 antibodies and express the corresponding antibody fragments in vivo.

As used herein, the term “Group 2 innate lymphoid cell” (ILC2) refers to a type of immune cell that plays a crucial role in the early stages of immune responses against parasitic worms, allergens, and other environmental insults. ILC2s are part of the innate immune system, which means that they can respond rapidly to a variety of stimuli without prior exposure or specific recognition of the pathogen. ILC2s are characterized by their ability to produce a variety of cytokines, including interleukin (IL)-5 and IL-13, which recruit and activate other immune cells, such as eosinophils, mast cells, and T helper 2 (Th2) cells, to promote a type 2 immune response. This type of response is important in combating parasitic infections, as well as in allergic reactions and tissue repair.

The term “type 2 inflammation” used herein also known as a type 2 immune response, is a specific type of immune response that is activated in response to a variety of stimuli, including parasitic infections, allergens, and tissue damage. Type 2 inflammation is characterized by the activation of immune cells such as Group 2 innate lymphoid cells (ILC2), eosinophils, mast cells, and T helper 2 (Th2) cells, which produce cytokines like interleukin (IL)-4, IL-5, and IL-13.

### The IL-25/IL-25R Axis

### I. IL-17 Cytokine Family

The IL-17 family is a group of cytokines that consists of six members: IL-17A, IL-17B, IL-17C, IL-17D, IL-17E (also known as IL-25), and IL-17F. These cytokines are produced by various types of immune cells, including T cells, mast cells, and innate lymphoid cells, and they play critical roles in both innate and adaptive immune responses (McGeachy, MJ Immunity. 2019 Apr. 16; 50(4): 892-906).

IL-17A and IL-17F are the most well-studied members of the IL-17 family, and they are often co-expressed and have similar functions. They promote inflammation by inducing the expression of pro-inflammatory cytokines, chemokines, and adhesion molecules, and by stimulating the recruitment and activation of neutrophils and other immune cells.

IL-17B, IL-17C, and IL-17E have more specialized roles. IL-17B and IL-17C are involved in host defense against bacterial and fungal infections, while IL-17E is important in allergic responses and protection against parasitic infections.

IL-17 cytokines interact with various cell types, that express different heterodimeric receptor complexes, comprised of five homologous subunits IL-17-RA to IL-17RE, with IL-17RA common to all receptors. Each subunit of the IL-17 receptor is a protein containing a single transmembrane domain with several conserved motifs, such as extracellular fibronectin III-like motifs, transmembrane regions, and cytoplasmic SEF/IL-17R (SEFIR) domains (Zhang Q. J Biol Chem. 2013; 288(51):36956-36965).

The IL-17 family cytokines have been implicated in the pathogenesis of various inflammatory and autoimmune diseases, including psoriasis, rheumatoid arthritis, multiple sclerosis, and inflammatory bowel disease, and they are targets for therapeutic intervention in these conditions.

### II. IL-25 (IL-17E)

IL-25 possesses approximately 23% to 33% homology with other IL-17 family members and binds to specific homologous IL-17 receptors to transmit signals Low sequence similarity of IL-25 to other family members suggests that it is highly unlikely that anti-IL25 specific antibodies bind to other IL-17 family members.

IL-25 is synthesized as a precursor molecule, which undergoes processing and cleavage to generate the mature, biologically active form of the cytokine. The cleavage of IL-25 precursor is mediated by several proteases, including furin and other proprotein convertases (PCs), which cleave the precursor protein at specific sites to generate the mature cytokine. The exact cleavage sites and mechanisms vary depending on the cell type or tissue in which it is produced. In particular, proteolytic cleavage of IL-25 at specific sites can either enhance or inhibit its ability to activate downstream signaling pathways (Matsushita, K. et al. JCI Insight 2020 Feb. 27; 5(4): e131480).

IL-25 is secreted as a disulfide-linked homodimer homodimeric glycoprotein, and is produced by a variety of cells, including epithelial cells, T cells, and innate lymphoid cells.

IL-25 interacts with, and signals through, the IL-25 receptor comprising heterodimeric IL-17RB/IL-17RA receptor subunits (Iwakura, et. al., (2010), Immunity, 34.149). Through its receptor, IL-25 promotes and sustains the Th2-type immune response and elicits the expansion of the type 2 innate lymphoid cells (ILC2) and M2 macrophages.

Although IL-25 is widely produced by various types of cells, the expression of its receptor is more limited. The IL-25 receptor is present on innate immune cells such as invariant natural killer T (iNKT) cells, ILC2s, eosinophils, basophils, mast cells, and antigen-presenting cells (APCs). Cell expression IL-17RB and how IL-17RB expression changes in different disorders, particularly in airway inflammatory disorders and skin inflammation (Rickel E A, J Immunol. 2008; 181(6):4299-4310).

IL-25 only interacts with IL-17RB to allosterically facilitate the formation of the ‘tip to tip’ interface of IL-17RB-IL-17RA, which is a key receptor-receptor interaction required to initiate signal transduction.

Upon binding to its receptor complex, IL-25 triggers the recruitment and phosphorylation of ACTI, which serves as a critical adapter protein for downstream signaling. IL-25 has been shown to activate several downstream signaling cascades, including nuclear factor kappa B (NF-kB), mitogen-activated protein kinases (MAPKs), and Janus kinase/signal transducer and activator of transcription (JAK/STAT) in a cell context-dependent manner (Borowczyk J et. Al., J Allergy Clin Immunol. July; 148(1):40-52 (2021), leading to the expression of various pro-inflammatory cytokines, chemokines, and other immune-related genes (Amini P., et al. (2018) American Journal of Rhinology & Allergy, 32(2), 115-124.).

### III. IL-25 Mechanism of Action in Pathogenesis

IL-25 may enhance a Th2-type immune response. Th2 cells are a subset of T cells that produce cytokines such as IL-4, IL-5, and IL-13, which are involved in allergic responses and defense against parasites. IL-25 can stimulate the differentiation of naïve T cells into Th2 cells and enhance the production of Th2 cytokines. This effect is important in the regulation of allergic responses and host defense against certain infections (Liu et al, J Immunol Res 2018.).

IL-25 may also stimulate innate lymphoid cells (ILC2s). ILCs are a group of immune cells that play a role in defense against pathogens and tissue repair. ILC2s are a subset of ILCs that produce Th2 cytokines and are involved in allergic responses. IL-25 can stimulate the activation and proliferation of ILC2s, leading to the production of Th2 cytokines and the initiation of immune responses (Wu. J. Front Immunol. 2022; 13: 986118).

IL-25 may also promote the accumulation of inducible costimulator (ICOS) and T1/ST2 on nuocytes. Nuocytes are a recently discovered subset of innate immune cells that produce Th2 cytokines and play a role in allergy and host defense. IL-25 can promote the accumulation of ICOS and T1/ST2, which are molecules expressed on the surface of nuocytes and involved in their activation and function (C. Wang, et al. PLoS One, vol. 11, no. 9).

IL-25 can also stimulate the differentiation of naïve T cells into Th2 cells, leading to the production of Th2 cytokines such as IL-4, IL-5, and IL-13.

Additionally, IL-25 increases production of chemokines and promotes the recruitment of eosinophils and inflammation. Chemokines regulate the migration and activation of immune cells IL-25 can increase the production of chemokines that promote the recruitment of eosinophils, which involved in the defense against parasites and allergic responses. This recruitment can lead to inflammation and tissue damage (J. Beale, et al Sci Trans Med, v6, no. 256, (2014).

IL-25 can also promote airway remodeling, which refers to the structural changes in the airways that occur in response to chronic inflammation. This can include increased mucus secretion, deposition of extracellular collagen, proliferation of smooth muscle cells, and angiogenesis (formation of new blood vessels). These changes can lead to airway obstruction and breathing (M. Suzukawa, et al., J Immunol. 2012 Oct. 1; 189(7)).

### IV. Therapeutic Targeting of IL-25-Related Diseases and Disorders

a. Type-2 Inflammatory Disorders

Respiratory system diseases like asthma and allergy are characterized by Type 2 inflammation. IL-25, a Type 2 cytokine produced by Th2 cells, induces the production of IL-4, IL-5, and IL-13, leading to respiratory tract inflammation.

Asthma is a heterogeneous inflammatory disease characterized by airflow obstruction, wheezing, eosinophilia and neutrophilia of the airways. Asthma can broadly be separated into two categories: eosinophilic (T-helper 2 [Th2]) and noneosinophilic (non-Th2), however multiple phenotypes, with varying underlying biology are recognized.

Elevated plasma levels of IL-25 are associated with the allergic asthma phenotype (Tang W., et al. Int Arch Allergy Immunol; 163: 5-10 (2014)), and IL-25 concentration in sputum correlates with disease severity. Sputum IL-25 is also increased in atopic versus non-atopic asthma patients (Paplinska-Goryca M. et al. Postepy Dermatol Alergol; 35: 462-469 (2018)). The relationship between IL-25 and neutrophilic airway inflammation suggests a pleiotropic role of IL-25 in the immune response in asthma. In addition, a rare allele of a component of the IL-25 receptor, IL-17RB, is associated with a reduced incidence of asthma (Jung J S, et al. Association of IL-17RB gene polymorphism with asthma. Chest 135: 1173-1180 (2009).

In animal models of asthma, overexpression or administration of recombinant IL-25 triggers allergic responses characterized by Th2 cytokine expression, eosinophilia, and mucus hypersecretion (U.S. Pat. No. 6,159,193). The effects of blocking TL-25 may be due to both reduction of Th2 cytokines in allergic responses and inhibition of expression of critical chemokines that promote an exacerbated inflammatory response (Petersen B C et al., Future Med Chem; 4:833-6 (2012)). Administration of anti-IL-25 antibodies has been shown to significantly reduce airway hyperreactivity, levels of Th2-associated cytokines, IgE levels, and goblet cell hyperplasia (Fort M M, et al., Immunity; 15:985-95 (2001), Rickel E A, et al., J Immunol; 181:4299-310 (2008), Ballantyne, S J et al., Journal of Allergy and Clinical Immunology:120 (6): 1324-1331 (2007)). Moreover, IL-25 knockout mice displayed reduced lung pathology in an asthma model (Ballantyne, S J, et al., Borowczyk J et. al., J Allergy Clin Immunol. July; 148(1):40-52 (2021)). These data indicate that targeting IL-25 or IL-17RB+ immune cells may be a promising strategy for the treatment of allergic inflammation, as shown in a preclinical study with ABM125, an anti-IL-25 monoclonal antibody (Lee, J., et al. Biochemical and biophysical research communications, 495(1), 1391-1397)).

Psoriasis is an autoimmune disease that causes skin inflammation, characterized by epidermal hyperplasia, increased angiogenesis, and dermal inflammation. While the exact cause of psoriasis is unknown, studies have suggested that an imbalance of Th1/Th2 cells and the involvement of Th17 cells may contribute to the disease. IL-25, a cytokine produced by keratinocytes, has been found to promote skin inflammation by recruiting neutrophils and activating macrophages. IL-25 is highly expressed in skin lesions of psoriasis patients and promotes the proliferation of IL-17RB+ keratinocytes, exacerbating the disease. Keratinocytes can be activated by IL-25 via activation of the STAT3 transcription factor, which leads to the expression of IL-17RB (Xu M, et al., Inflammation. Immunity (2018) 48(4):787-98)). Blockade of IL-17RA, a co-receptor for TL-17A, IL-17F, and IL-25, has shown high efficacy in the treatment of psoriasis, suggesting that blocking IL-25 may be a promising strategy for targeting skin inflammation.

b. Autoimmune Diseases

Studies have suggested that IL-25 may have both pro-inflammatory and anti-inflammatory effects, depending on the specific disease context and the cells involved. In some autoimmune diseases, such as Rheumatoid arthritis (RA) and Multiple sclerosis (MS), IL-25 has been found to have a protective effect as an anti-inflammatory cytokine and an inhibitor of both innate and adaptive immunity. In some diseases such as Inflammatory Bowel Disease (IBD) and Systemic Lupus Erythematosus (SLE), IL-25 has a dual role both pro-inflammatory and anti-inflammatory effects in regulating immune responses during the development of diseases. (Deng, D., et. al. Front Immunol. 2021; 12: 691559).

However, in other autoimmune diseases such as Sjogren's Syndrome (SS), studies have indicated that IL-25 plays a pathogenic role during the development of SS. Studies have shown that IL-17A plays a key role in the pathogenesis of Sjogren's syndrome by promoting the activity of Th17 cells. Recently, it was observed that IL-25 is significantly increased in SG and peripheral blood of SS patients and promotes the activation of inflammatory ILC2s. Blocking IL-25 using a neutralizing antibody improves saliva flow rate and SG tissue damage in mice with experimental SS, accompanied by decreased ILC2 infiltration. Upregulation of TRAF6 in CD3+ T cells and ILC2s in SGs of pSS patients suggests that IL-25 signals are functional via coordinating activation of ERK1/2 and relative transcription factors (Guggino G, Arthritis Rheumatol (2018) 70(8):1265-75).

Overall, the impact of IL-25 on autoimmune diseases is complex and context-dependent, and further research is needed to fully understand its role in these diseases and to explore its potential as a therapeutic target. Blocking IL-25 may be a promising strategy for targeting autoimmune diseases such as Sjogren's Syndrome.

c. Cancer

IL-25 has been shown to have potential anticancer effects by modulating immune responses and inhibiting tumor growth (Shabgah. A, et al Cancer Med. 2021 August; 10(15): 5191-5202).

IL-25 can stimulate the activity of immune cells such as natural killer (NK) cells, dendritic cells, and CD8+ T cells, which can directly kill cancer cells. It can also promote the production of other cytokines that enhance immune cell activity, such as interferon-gamma (IFN-7) and tumor necrosis factor-alpha (TNF-α) (Sfanos, K. S., et. al. (2014). Clinical cancer research, 20(12), 3254-3265).

In addition to its immune-boosting effects, IL-25 has been found to have direct antitumor effects in various cancer types. For example, IL-25 has been shown to induce apoptosis (programmed cell death) in prostate cancer cells, and to inhibit the growth of colon cancer cells by blocking cell cycle progression (Zhang, J., et. al (2019). Oncology letters, 18(5), 5179-5186).

Furthermore, IL-25 has been found to enhance the efficacy of chemotherapy and radiation therapy in preclinical models of cancer. IL-25 treatment has been shown to sensitize cancer cells to chemotherapy by increasing their susceptibility to cell death, and to enhance the effects of radiation therapy by stimulating immune responses and reducing tumor blood vessel density (Bao, H et. al. (2017). Scientific reports, 7(1), 1-11).

Taken together, these findings suggest that IL-25 has promising potential as an anticancer agent, either alone or in combination with other treatments. However, further research is needed to fully understand the mechanisms underlying its antitumor effects and to explore its potential as a therapeutic agent in clinical settings.

### Anti-IL-25 Antibodies

The anti-IL-25 antibodies of the disclosure (e.g., IL25Ab1, IL25Ab2, IL25Ab3, IL25Ab4, IL25Ab5, IL25Ab6, IL25Ab7, IL25Ab8, IL25Ab9, IL25Ab10 and IL25Ab11) bind (e.g., specifically bind) human IL-25. Such antibodies and fragments thereof may be useful to disrupt the IL-25/IL-25R binding interaction. These antibodies and fragments thereof are characterized by unique sets of CDR sequences, specificity for IL-25 and are useful in treating Type 2 inflammation diseases, autoimmune diseases, or cancer as a monotherapy or in combination with other agents. More specifically, the disclosure relates to antibodies and fragments thereof that bind to human IL-25, and to their use to modulate an IL-25-mediated activity of cells localized to an inflammation disease microenvironment or a tumor microenvironment.

In an embodiment, the anti-IL-25 antibody or antibody fragment thereof comprises a heavy chain variable region (VH) having a set of CDRs (HCDR1, HCDR2, and HCDR3) disclosed in Table 1. For example, the anti-IL-25 antibody or antibody fragment thereof may comprise a set of CDRs corresponding to those CDRs in one or more of the anti-IL-25 antibodies disclosed in Table 1 (e.g., the HCDRs of the IL25Ab1 antibody).

In another embodiment, the anti-IL-25 antibody or fragment thereof comprises a light chain variable region (VL) having a set of CDRs (LCDR, LCDR2, and LCDR3) as disclosed in Table 2. For example, the anti-IL-25 antibody or antibody fragment thereof may comprise a set of CDRs corresponding to those CDRs in one or more of the anti-IL-25 antibodies disclosed in Table 2 (e.g., the LCDRs of the L25Ab1 antibody).

In an alternative embodiment, the anti-IL-25 antibody or antibody fragment thereof comprises a VH having a set of CDRs (HCDR1, HCDR2, and HCDR3) as disclosed in Table 1, and a VL having a set of CDRs (LCDR1, LCDR2, and LCDR3) as disclosed in Table 2.

TABLE 1
CDR Sequences of Murine Heavy Chain Variable (VH) Domains

| Anti-IL-25 Ab | CDR1 | CDR2 | CDR3 |
|---|---|---|---|
| IL25Ab1 | SEQ ID NO: 11 | SEQ ID NO: 12 | SEQ ID NO: 13 |
| IL25Ab2 | SEQ ID NO: 17 | SEQ ID NO: 18 | SEQ ID NO: 19 |
| IL25Ab3 | SEQ ID NO: 23 | SEQ ID NO: 24 | SEQ ID NO: 25 |
| IL25Ab4 | SEQ ID NO: 29 | SEQ ID NO: 30 | SEQ ID NO: 31 |
| IL25Ab5 | SEQ ID NO: 35 | SEQ ID NO: 36 | SEQ ID NO: 37 |

TABLE 2
CDR Sequences of Murine Light Chain Variable (VL) Domains

| Anti-IL-25 Ab | CDR1 | CDR2 | CDR3 |
|---|---|---|---|
| IL25Ab1 | SEQ ID NO: 14 | SEQ ID NO: 15 | SEQ ID NO: 16 |
| IL25Ab2 | SEQ ID NO: 20 | SEQ ID NO: 21 | SEQ ID NO: 22 |
| IL25Ab3 | SEQ ID NO: 26 | SEQ ID NO: 27 | SEQ ID NO: 28 |
| IL25Ab4 | SEQ ID NO: 32 | SEQ ID NO: 33 | SEQ ID NO: 34 |
| IL25Ab5 | SEQ ID NO: 38 | SEQ ID NO: 39 | SEQ ID NO: 40 |

In an embodiment, the anti-IL-25 antibody or antibody fragment thereof comprises a humanized heavy chain variable region (VH) having a set of CDRs (HCDR1, HCDR2, and HCDR3) disclosed in Table 3. For example, the anti-IL-25 antibody or antibody fragment thereof may comprise a set of CDRs corresponding to those CDRs in one or more of the anti-IL-25 antibodies disclosed in Table 1 (e.g., the HCDRs of the IL25Ab6 antibody).

In another embodiment, the anti-IL-25 antibody or fragment thereof comprises a humanized light chain variable region (VL) having a set of CDRs (LCDR1, LCDR2, and LCDR3) as disclosed in Table 4. For example, the anti-IL-25 antibody or antibody fragment thereof may comprise a set of CDRs corresponding to those CDRs in one or more of the anti-IL-25 antibodies disclosed in Table 2 (e.g., the LCDRs of the IL25Ab6 antibody).

In an alternative embodiment, the anti-IL-25 antibody or antibody fragment thereof comprises a VH having a set of CDRs (HCDR1, HCDR2, and HCDR3) as disclosed in Table 3, and a VL having a set of CDRs (LCDR1, LCDR2, and LCDR3) as disclosed in Table 4.

TABLE 3
CDR Sequences of Humanized Heavy
Chain Variable (VH)Domains

| Anti-IL-25 Ab | CDR1 | CDR2 | CDR3 |
|---|---|---|---|
| IL25Ab6/IL25Ab7/ | SEQ ID | SEQ ID | SEQ ID NO: 25 |
| IL25Ab9/IL25Ab10 | NO: 23 | NO: 49 | |
| IL25Ab8/IL25Ab11 | SEQ ID | SEQ ID | SEQ ID NO: 25 |
| | NO: 23 | NO: 52 | |

TABLE 4
CDR Sequences of Humanized Light
Chain Variable (VL) Domains

| Anti-IL-25 Ab | CDR1 | CDR2 | CDR3 |
|---|---|---|---|
| IL25Ab6/IL25Ab7/ | SEQ ID | SEQ ID | SEQ ID NO: 28 |
| IL25Ab8/IL25Ab9/ | NO: 50 | NO: 51 | |
| IL25Ab10/IL25Ab11 | | | |

In an embodiment, the anti-IL-25 antibody or antibody fragment thereof comprises a VH having a set of complementarity-determining regions (HCDR1, HCDR2, and HCDR3) selected from the group consisting of:
(i) HCDR1: SEQ ID NO: 11, HCDR2: SEQ ID NO: 12, HCDR3: SEQ ID NO: 13;
(ii) HCDR1: SEQ ID NO: 17, HCDR2: SEQ ID NO: 18, HCDR3: SEQ ID NO: 19;
(iii) HCDR1: SEQ ID NO: 23, HCDR2: SEQ ID NO: 24, HCDR3: SEQ ID NO: 25;
(iv) HCDR1: SEQ ID NO: 29, HCDR2: SEQ ID NO: 30, HCDR3: SEQ ID NO: 31;
(v) HCDR1: SEQ ID NO: 35, HCDR2: SEQ ID NO: 36, HCDR3: SEQ ID NO: 37;
(vi) HCDR1: SEQ ID NO: 23, HCDR2: SEQ ID NO: 49, HCDR3: SEQ ID NO: 25 and
(vii) HCDR1: SEQ ID NO: 23 HCDR2: SEQ ID NO: 52, HCDR3: SEQ ID NO: 25.

In another embodiment, the anti-IL-25 antibody or antibody fragment thereof comprises a VL having a set of complementarity-determining regions (LCDR1, LCDR2, and LCDR3) selected from the group consisting of:
(i) LCDR1: SEQ ID NO: 14, LCDR2: SEQ ID NO: 15, LCDR3: SEQ ID NO 16;
(ii) LCDR1: SEQ ID NO: 20, LCDR2: SEQ ID NO: 21, LCDR3: SEQ ID NO: 22;
(iii) LCDR1: SEQ ID NO: 26, LCDR2: SEQ ID NO: 27, LCDR3: SEQ ID NO: 28;
(iv) LCDR1: SEQ ID NO: 32, LCDR2: SEQ ID NO: 33, LCDR3: SEQ ID NO: 34;
(v) LCDR1: SEQ ID NO 38, LCDR2: SEQ ID NO: 39, LCDR3 SEQ ID NO: 40; and
(vi) LCDR1: SEQ ID NO: 50, LCDR2: SEQ ID NO: 51, LCDR3: SEQ ID NO: 28.

In another embodiment, the anti-IL-25 antibody or antibody fragment thereof comprises:
(a) a VH having a set of complementarity-determining regions (HCDR1, HCDR2, and HCDR3) selected from the group consisting of:
   (i) HCDR1: SEQ ID NO: 11, HCDR2: SEQ ID NO: 12, HCDR3: SEQ ID NO: 13;
   (ii) HCDR1: SEQ ID NO: 17, HCDR2 SEQ ID NO: 18, HCDR3: SEQ ID NO:19;
   (iii) HCDR1: SEQ ID NO: 23, HCDR2: SEQ ID NO: 24, HCDR3: SEQ ID NO: 25;
   (iv) HCDR1: SEQ ID NO: 29, HCDR2: SEQ ID NO: 30, HCDR3: SEQ ID NO: 31;
   (v) HCDR1: SEQ ID NO: 35, HCDR2: SEQ ID NO: 36, HCDR3: SEQ ID NO: 37;
   (vi) HCDR1: SEQ ID NO: 23, HCDR2: SEQ ID NO: 49, HCDR3: SEQ ID NO: 25 and
   (vii) HCDR1: SEQ ID NO: 23 HCDR2: SEQ ID NO: 52, HCDR3: SEQ ID NO: 25.
(b) a VL having a set of complementarity-determining regions (LCDR1, LCDR2, and LCDR3) selected from the group consisting of:
   (i) LCDR1: SEQ ID NO: 14, LCDR2: SEQ ID NO: 15, LCDR3: SEQ ID NO: 16;
   (ii) LCDR1: SEQ ID NO 20, LCDR2: SEQ ID NO: 21, LCDR3 SEQ ID NO: 22;
   (iii) LCDR1: SEQ ID NO: 26, LCDR2: SEQ ID NO: 27, LCDR3: SEQ ID NO: 28;
   (iv) LCDR1: SEQ ID NO: 32, LCDR2: SEQ ID NO: 33, LCDR3: SEQ ID NO: 34;
   (v) LCDR1: SEQ ID NO: 38, LCDR2: SEQ ID NO: 39, LCDR3: SEQ ID NO: 40;
   (vi) LCDR1: SEQ ID NO: 50, LCDR2: SEQ ID NO: 51, LCDR3: SEQ ID NO: 28.

In an embodiment, the antibody or antibody fragment thereof comprises a combination of a VH and a VL having a set of complementarity-determining regions (CDR1, CDR2 and CDR3) selected from the group consisting of:
(i) VH: CDR1: SEQ ID NO: 11, CDR2: SEQ ID NO: 12, CDR3: SEQ ID NO: 13, VL: CDR1: SEQ ID NO: 14, CDR2: SEQ ID NO: 15, CDR3: SEQ ID NO: 16;
(ii) VH: CDR1: SEQ ID NO: 17, CDR2: SEQ ID NO: 18, CDR3: SEQ ID NO: 19, VL: CDR1: SEQ ID NO: 20, CDR2: SEQ ID NO: 21, CDR3: SEQ ID NO: 22;
(iii) VH: CDR1: SEQ ID NO: 23, CDR2: SEQ ID NO: 24, CDR3: SEQ ID NO: 25, VL: CDR1: SEQ ID NO: 26, CDR2: SEQ ID NO: 27, CDR3: SEQ ID NO: 28;
(iv) VH: CDR1: SEQ ID NO: 29, CDR2: SEQ ID NO: 30, CDR3: SEQ ID NO: 31, VL: CDR1: SEQ ID NO: 32, CDR2: SEQ ID NO: 33, CDR3: SEQ ID NO: 34;
(v) VH: CDR1: SEQ TD NO: 35, CDR2 SEQ ID NO: 36, CDR3: SEQ ID NO: 37, VL: CDR1: SEQ ID NO: 38, CDR2: SEQ ID NO: 39, CDR3: SEQ ID NO: 40;
(vi) VH: CDR1: SEQ ID NO: 23, CDR2: SEQ ID NO: 49, CDR3: SEQ ID NO: 25, VL: CDR1: SEQ ID NO: 50, CDR2: SEQ ID NO: 51, CDR3: SEQ ID NO: 28; and
(vii) VH: CDR1: SEQ ID NO: 23, CDR2: SEQ ID NO: 52, CDR3: SEQ ID NO: 25, VL: CDR1: SEQ ID NO: 50, CDR2: SEQ ID NO:51, CDR3: SEQ ID NO: 28.

In an embodiment, the anti-IL-25 antibody or antibody fragment thereof comprises a variable heavy chain sequence selected from the group consisting of: SEQ ID NOs: 1, 3, 5, 7, 9, 44 and 47; and/or a variable light chain sequence selected from the group consisting of: SEQ ID NOs: 2, 4, 6, 8, 10, 45, 46 and 48.

In an embodiment, the anti-IL-25 antibody or antibody fragment thereof comprises a pair of variable heavy chain and variable light chain sequences, selected from the following combinations: a variable heavy chain sequence comprising SEQ ID NO: 1 and a variable light chain sequence comprising SEQ ID NO: 2; a variable heavy chain sequence comprising SEQ ID NO: 3 and a variable light chain sequence comprising SEQ ID NO: 4; a variable heavy chain sequence comprising SEQ ID NO: 5 and a variable light chain sequence comprising SEQ ID NO: 6; and a variable heavy chain sequence comprising SEQ ID NO: 7 a variable light chain sequence comprising SEQ ID NO: 8; a variable heavy chain sequence comprising SEQ ID NO: 9 a variable light chain sequence comprising SEQ ID NO: 10; a variable heavy chain sequence comprising SEQ ID NO: 44 and a variable light chain sequence comprising SEQ ID NO: 45; a variable heavy chain sequence comprising SEQ ID NO: 44 and a variable light chain sequence comprising SEQ ID NO: 46; and a variable heavy chain sequence comprising SEQ ID NO: 47 and a variable light chain sequence comprising SEQ ID NO: 48. The skilled person will further understand that the variable light and variable heavy chains may be independently selected, or mixed and matched, to prepare an anti-IL-25 antibody or fragment thereof comprising a combination of variable heavy and variable light chain that is distinct from the pairings identified above.

In an embodiment, the anti-IL-25 antibody or antibody fragment thereof comprises a pair of variable heavy chain and variable light chain sequences, selected from the following combinations: a variable heavy chain sequence that is 90%, 95%, or 99% identical to SEQ ID NO: 1 and a variable light chain sequence that is 90%, 95%, or 99% identical to SEQ ID NO: 2; a variable heavy chain sequence that is 90%, 95%, or 99% identical to SEQ ID NO: 3 and a variable light chain sequence that is 90%, 95%, or 99% identical to SEQ ID NO: 4; a variable heavy chain sequence that is 90%, 95%, or 99% identical to SEQ ID NO: 5 and a variable light chain sequence that is 90%, 95%, or 99% identical to SEQ ID NO: 6; a variable heavy chain sequence that is 90%, 95%, or 99% identical to SEQ ID NO: 7 and a variable light chain sequence that is 90%, 95%, or 99% identical to SEQ ID NO: 8; a variable heavy chain sequence that is 90%, 95%, or 99% identical to SEQ ID NO: 9 and a variable light chain sequence that is 90%, 95%, or 99% identical to SEQ ID NO: 10; a variable heavy chain sequence that is 90%, 95%, or 99% identical to SEQ ID NO: 44 and a variable light chain sequence that is 90%, 95%, or 99% identical to SEQ ID NO: 45; a variable heavy chain sequence that is 90%, 95%, or 99% identical to SEQ ID NO: 44 and a variable light chain sequence that is 90%, 95%, or 99% identical to SEQ ID NO: 46; and a variable heavy chain sequence that is 90%, 95%, or 99% identical to SEQ ID NO: 47 and a variable light chain sequence that is 90%, 95%, or 99% identical to SEQ ID NO: 48. The skilled person will further understand that the variable light and variable heavy chains may be independently selected, or mixed and matched, to prepare an anti-IL-25 antibody or antibody fragment thereof comprising a combination of variable heavy and variable light chain that is distinct from the pairings identified above.

In some embodiments, the anti-TL-25 antibody or antibody fragment thereof comprises one or more conservative amino acid substitutions. A person of skill in the art will recognize that a conservative amino acid substitution is a substitution of one amino acid with another amino acid that has similar structural or chemical properties, such as, for example, a similar side chain. Exemplary conservative substitutions are described in the art, for example, in Watson et al., Molecular Biology of the Gene, The Benjamin/Cummings Publication Company, 4th Ed. (1987).

“Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al. (1998) Acta Physiol Sc and Suppl 643: 55-67; Sasaki et al. (1998) Adv Biophys 35: 1-24). Amino acid substitutions to the antibodies of the invention may be made by known methods for example by PCR mutagenesis (U.S. Pat. No. 4,683,195).

In some embodiments, the antibody or fragment thereof comprises a variable heavy chain sequence that comprises an amino acid sequence with at least about 95%, about 96%, about 97%, about 98%, or about 99%, sequence identity to the amino acid sequence set forth in SEQ ID NOs: 1, 3, 5, 7, 9, 44 or 47. In other embodiments, the antibody or fragment thereof retains the binding and/or functional activity of an antibody or fragment thereof that comprises the variable heavy chain sequence of SEQ ID NOs: 1, 3, 5, 7, 9, 44 or 47. In still further embodiments, the antibody or fragment thereof comprises the variable heavy chain sequence of SEQ ID NOs: 1, 3, 5, 7, 9, 44 or 47 and have one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NOs: 1, 3, 5, 7, or 9, 44, or 47 (based on the numbering system of Kabat).

In particular embodiments, the antibody or fragment thereof comprises a variable heavy chain sequence with at least about 95%, about 96%, about 97%, about 98%, or about 99% sequence identity to the antibody or fragment thereof heavy chain variable region sequence set forth in SEQ ID NOs: 1, 3, 5, 7, 9, 44, or 47 comprises one or more conservative amino acid substitutions in a framework region and retains the binding and/or functional activity of an antibody or fragment thereof that comprises a variable heavy chain sequence as set forth in SEQ ID NOs: 1, 3, 5, 7, 9, 44, or 47 and a variable light chain sequence as set forth in SEQ ID NOs: 2, 4, 6, 8, 10, 45, 46, or 48.

In some embodiments, the antibody or fragment thereof comprises a variable light chain sequence that comprises an amino acid sequence with at least about 95%, about 96%, about 97%, about 98%, or about 99%, sequence identity to the amino acid sequence set forth in SEQ ID NOs: 2, 4, 6, 8, 10, 45, 46, or 48.

In other embodiments, the antibody or fragment thereof retains the binding and/or functional activity of an antibody or fragment thereof that comprises the variable light chain sequence of SEQ ID NOs: 2, 4, 6, 8, 10, 45, 46, or 48.

In still further embodiments, the antibody or fragment thereof comprises the variable light chain sequence of SEQ ID NOs: 2, 4, 6, 8, 10, 45, 46, or 48 and have one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NOs: 2, 4, 6, 8, 10, 45, 46, or 48 (based on the numbering system of Kabat).

In particular embodiments, the antibody or fragment thereof comprises a variable light chain sequence with at least about 95%, about 96%, about 97%, about 98%, or about 99% sequence identity to the antibody or fragment thereof light chain variable region sequence set forth in SEQ ID NOs: 2, 4, 6, 8, 10, 45, 46, or 48.

In some embodiments, the anti-IL25 antibody comprises one or more conservative amino acid substitutions in a framework region and retains the binding and/or functional activity of an antibody or fragment thereof that comprises a variable heavy chain sequence as set forth in SEQ ID NOs: 1, 3, 5, 7, 9, 44, or 47 and a variable light chain sequence as set forth in SEQ ID NOs: 2, 4, 6, 8, 10, 45, 46, or 48.

In some embodiments, the anti-IL-25 antibody is a monoclonal antibody. In some embodiments, the anti-IL-25 antibody is a human antibody, or antigen-binding portions thereof. In alternative embodiments, the anti-IL-25 antibody is a murine antibody or a chimeric antibody. In some embodiments, the anti-IL-25 antibody is a humanized antibody. In some embodiments, the anti-IL-25 antibody is a fully human antibody. In an alternative embodiment, an antibody fragment comprising the antigen-binding portion of one or more of the disclosed anti-IL-25 antibodies may be incorporated into a bi- or multi-specific antibody or into a fusion protein.

In one embodiment, the anti-IL-25 antibody or antibody fragment thereof comprises all six of the murine CDR regions of the IL25Ab1, IL25Ab2, IL25Ab3, IL25Ab4 or IL25Ab5 antibodies formatted as a chimeric or a humanized antibody. In other embodiments, the anti-IL-25 antibody or antibody fragment thereof comprises all six of the CDR regions of one of the disclosed murine antibodies.

In some embodiments, the anti-IL-25 antibody is a humanized antibody (e.g., IL25Ab6, IL25Ab7, IL25Ab8, IL25Ab9, IL25Ab10 or IL25Ab11) comprising the VH/VL CDR regions set forth in Tables 3 and 4.

In some embodiments, the antibody is a full-length antibody. In other embodiments, the antibody is an antibody fragment including, for example, an antibody fragment selected from the group consisting of: Fab, Fab′, F(ab)2, Fv, domain antibodies (dAbs), and complementarity determining region (CDR) fragments, single-chain antibodies (scFv), chimeric antibodies, diabodies, triabodies, tetrabodies, miniantibodies, and polypeptides that contain at least a portion of an immunoglobulin that is sufficient to confer IL-25 specific binding to the polypeptide.

In some embodiments, a variable region domain of an anti-IL-25 antibody disclosed herein may be covalently attached at a C-terminal amino acid to at least one other antibody domain or a fragment thereof. Thus, for example, a VH domain that is present in the variable region domain may be linked to an immunoglobulin CH1 domain, or a fragment thereof. Similarly, a VL domain may be linked to a CK domain or a fragment thereof. In this way, for example, the antibody may be a Fab fragment wherein the antigen binding domain contains associated VH and VL domains covalently linked at their C-termini to a CH1 and CK domain, respectively. The CH1 domain may be extended with further amino acids, for example to provide a hinge region or a portion of a hinge region domain as found in a Fab fragment, or to provide further domains, such as antibody CH2 and CH3 domains.

In some embodiments, a variable region domain of an anti-IL-25 antibody disclosed herein may be covalently attached at a C-terminal amino acid to an antibody constant region. For example, a VL domain may be linked to the mouse Kappa constant region (SEQ ID NO: 54) or the human Kappa constant region (SEQ ID NO: 57). Similarly, a VH domain may be linked to the mouse IgG1 constant region (SEQ ID NO: 53), human IgG1 constant region (SEQ ID NO: 55), or another antibody constant region such as human IgG4 or IgG2. The constant regions may contain certain mutations to modulate the properties of a derived antibody. For example, a triple mutation YTE may be introduced into human IgG1 constant region (SEQ ID NO: 56) to extend the antibody half-life. Fc Mutations that modulate the effector functions of antibody may also be introduced.

Thus, in one embodiment, the antibody fragment comprises at least one CDR as described herein. The antibody fragment may comprise at least two, three, four, five, or six CDRs as described herein. The antibody fragment further may comprise at least one variable region domain of an antibody described herein. The variable region domain may be of any size or amino acid composition and will generally comprise at least one CDR sequence responsible for binding to human IL-25, for example, CDR-H1, CDR-H2, CDR-H3, CDR-L1, CDR-L2, and/or CDR-L3 as described herein, and which is adjacent to or in frame with one or more framework sequences.

In a further aspect, the anti-IL-25 antibody or antibody fragment thereof exhibits one or more of the following properties:
(a) is specific for human IL-25 and has the ability to block IL-25 binding to its receptor;
(b) inhibits, interferes with, or modulates IL-25 interaction with IL-25 receptor signal transduction;
(c) inhibits intracellular NF-kB activation induced by IL-25;
(d) inhibits 11-5 production induced by human TL-25 in human PBMC;
(e) inhibits CXCL-1 production induced by a human IL-25 in a human cell colon cancer cell line;
(f) binds to mouse and cynomolgus IL-25;
(g) reduces IL-5 production in the lung of OVA-induced asthma model; and
(h) improves airway resistance in the OVA-induced asthma model.

In an embodiment, the anti-IL-25 antibodies or antibody fragments thereof can reduce, inhibit, interfere with, and/or modulate at least one of the biological responses related to IL-25, and as such, are useful for ameliorating the effects of IL-25-related diseases or disorders. Such antibodies and antibody fragments thereof can be used, for example, to reduce, inhibit, interfere with and/or modulate IL-25 signaling, IL-25 activation of epithelial cells and type 2 lymphocytes, IL-25 activation of tumor cells, or induce production of proinflammatory cytokines.

The disclosed antibodies (e.g., IL25Ab1, IL25Ab2, IL25Ab3, IL25Ab4, IL25Ab5, IL25Ab6, IL25Ab7, IL25Ab8, IL25Ab9, IL25Ab10, and IL25Ab11) bind (e.g., specifically bind) to human IL25. The amino acid sequences of the closely related human IL-17 family members (IL-17A (Q16552), IL-17B (Q9UHF5), IL-17C (Q9POM4), IL-17D (Q8TAD2) and IL-17F (Q96PD4)) were retrieved from UniProt. Human IL-25 exhibits low homology to its phylogenetically close relatives. IL-25 shows very low sequence similarity to other family members ranging from 23.2% to 32.7%. Based on the low homology, it is unlikely that anti-IL25 antibodies will bind to IL17 family members.

Antibodies typically bind specifically to their cognate antigen with high affinity, reflected by a dissociation constant (KD) of 10⁻⁷ to 10⁻¹¹ M or less. Any KD greater than about 10⁻⁶ M is generally considered to indicate nonspecific binding. As used herein, an antibody that “binds specifically” to an antigen refers to an antibody that binds to the antigen and substantially identical antigens with high affinity, which means having a KD of 10⁻⁷ M or less, preferably 10⁻⁸ M or less, even more preferably 5×10⁻⁹ M or less, and most preferably between 10⁻⁸ M and 10⁻¹⁰ M or less but does not bind with high affinity to an unrelated antigen. The disclosed antibodies bind to the human IL25 with high affinity, with KD determined by BLI<1.0E-12.

The term “cross-reacts,” as used herein, refers to the ability of anti-human IL-25 specific antibody described herein to bind to IL25 from a different species. For example, an antibody described herein may also bind IL25 from another species (e.g., cynomolgus monkey or mouse 11.25). As used herein, cross-reactivity may be measured by detecting a specific reactivity with purified antigen in binding assays (e.g., SPR, ELISA) or binding to, or otherwise functionally interacting with, cells physiologically expressing IL25. Methods for determining cross-reactivity include standard binding assays as described herein, for example, by BIACORE® surface plasmon resonance (SPR) analysis using a BIACORE® 2000 SPR instrument (Biacore AB, Uppsala, Sweden), Biolayer interferometry (BLI), or flow cytometric techniques.

The disclosed human IL2-5 antibodies IL25Ab1 to IL25Ab11, all bind to IL-25 from cynomolgus monkey with notable affinity. IL25Ab1 to IL25Ab11 bind to cyno IL-25 and mouse IL-25 in ELISA assays with affinities comparable to the binding affinity to human IL-25.

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antibody fragments thereof.

Antibodies may be prepared as chimeric antibodies or antibody fragments thereof with murine variable regions and human constant regions. Human antibody constant regions may be of reported allotypes, reviewed in Jefferis et al., Human immunoglobulin allotypes: possible implications for immunogenicity. MAbs 1, 332-338 (2009. In one embodiment, the human heavy chain constant region uses a consensus human IgG1 constant region sequence (SEQ ID NO 55) whereas the light chain constant region uses a consensus human kappa constant region sequence (SEQ ID NO: 57). Human IgG1 may be chosen because it is one of the most common subtype for chimera antibody generation and can provide effector function. Human kappa constant region may be used because a majority of parental murine antibodies are of mouse kappa light chain.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore and/or improve antibody binding specificity or affinity. In some embodiments, some CDR residues in a humanized antibody (e.g, 1, 2, 3, 4, or 5 residues) may be mutated to improve antibody developability.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008) and framework regions derived from screening FR libraries (see, e.g., Baca et al., J. Biol. Chem. 272:10678-10684 (1997) and Rosok et al., J. Biol. Chem. 271:22611-22618 (1996)).

For human or humanized antibodies such as IgG1 antibody, amino acid substitutions have been introduced into the Fc region to modulate the antibody interactions with Fc receptors, FcRn, or complement, resulting in the modulated antibody effector functions and/or antibody half-life (see, e.g., Damelang, T., et al. “Impact of structural modifications of IgG antibodies on effector functions.” Front Immunol 14: 1304365 (2023). Among the Fe mutations that extend antibody half-life, YTE (M252Y, S254T and T256E in EU numbering) and LS (M428L and N434S) are two examples used in the literature.

### Methods of Producing IL-25 Antibodies

An anti-IL-25 antibody or antibody fragment thereof may be made by any method known in the art. For example, a recipient may be immunized with soluble recombinant human IL-25 protein, or a fragment or a peptide conjugated with a carrier protein thereof. Any suitable method of immunization can be used. Such methods can include adjuvants, other immune stimulants, repeat booster immunizations, and the use of one or more immunization routes.

Any suitable source of human IL-25 can be used as the immunogen for the generation of the anti-IL-25 antibodies of the compositions and methods disclosed herein. Different forms of the IL-25 antigen may be used to generate the antibody that is sufficient to generate a biological activity. Thus, the eliciting IL-25 antigen may be a single epitope, multiple epitopes, or the entire protein alone or in combination with one or more immunogenicity enhancing agents. In some aspects, the eliciting antigen is an isolated soluble full-length protein, or a soluble protein comprising less than the full-length sequence (e.g., immunizing with a peptide comprising particular portion or epitopes of TL-25). As used herein, the term “portion” refers to the minimal number of amino acids or nucleic acids, as appropriate, to constitute an immunogenic epitope of the antigen of interest. Any genetic vectors suitable for transformation of the cells of interest may be employed, including, but not limited to adenoviral vectors, plasmids, and non-viral vectors, such as cationic lipids.

It is desirable to prepare monoclonal antibodies (mAbs) from various mammalian hosts, such as mice, rodents, primates, humans, etc. Description of techniques for preparing such monoclonal antibodies may be found in, e.g., Sties et al. (eds.) BASIC AND CLINICAL IMMUNOLOGY (4th ed.) Lance Medical Publication, Los Altos, CA, and references cited therein; Harlow and Lane (1988) ANTIBODIES: A LABORATORY MANUAL CSH Press; Goding (1986) MONOCLONAL ANTIBODIES: PRINCIPLES AND PRACTICE (2nd ed.) Academic Press, New York, NY. Typically, spleen cells from an animal immunized with a desired antigen are immortalized, commonly by fusion with a myeloma cell. See Kohler and Milstein (196) Eur. J. Immunol. 6:511-519. Alternative methods of immortalization include transformation with Epstein Barr Virus, oncogene, or retroviruses, or other methods known in the art. See, e.g., Doyle et al. (eds. 1994 and periodic supplements) CELL AND TISSUE CULTURE: LABORATORY PROCEDURES, John Wiley and Sons, New York, NY. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or an antigen binding fragment thereof by screening a DNA library from human B cells according, e.g., to the general protocol outlined by Huse et al., (1989) Science 246: 1275-1281. Thus, antibodies may be obtained by a variety of techniques familiar to researchers skilled in the art.

Other suitable techniques involve selection of libraries of antibodies in phage, yeast, virus or similar vector. See e.g., Huse et al., supra; and Ward et al., (1989) Nature 341:544-546. The polypeptides and antibodies disclosed herein may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literatures. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,996,345; 4,277,437; 4,275,149; and 4,366,241. Also, recombinant immunoglobulins may be produced, see Cabilly U.S. Pat. No. 4,816,567; and Queen et al. (1989) Proc. Nat'l Acad. Sci. USA 86: 10029-10023; or made in transgenic mice, see Nils Lonberg et al., (1994), Nature 368:856-859; and Mendez et al. (1997) Nature Genetics 15: 146-156; TRANSGENIC ANIMALS AND METHODS OF USE (WO 2012/62118), Medarex, Trianni, Abgenix, Ablexis, OminiAb, Harbour and other technologies.

In some embodiments, the ability of the produced antibody to bind to IL-25 can be assessed using standard binding assays, such as surface plasmon resonance (SPR), Octet (BLI), ELISA, Western Blot, immunofluorescence, flow cytometric analysis, chemotaxis assays, and cell migration assays. In some aspects, the produced antibody may also be assessed for its ability to inhibit IL-25 from blocking IL-25 receptor signal transduction and inhibit IL-25-mediated inflammatory microenvironment successive effects including inhibiting IL-25 induced NFkB signaling, IL-5 production and/or CXCL1 production.

The antibody composition prepared from the cells can be purified using, for example, hydroxylapatite chromatography, gel electrophoresis, dialysis, and affinity chromatography, with affinity chromatography being a typical purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (see, e.g., Lindmark et al., 1983 J. Immunol. Meth. 62:1-13). Protein G is recommended for all mouse isotypes and for human γ3 (see, e.g., Guss et al., 1986 EMBO J. 5:1567-1575). A matrix to which an affinity ligand is attached is most often agarose, but other matrices are available. Mechanically stable matrices such as controlled pore glass or poly (styrenedivinyl) benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, typically performed at low salt concentrations (e.g., from about 0-0.25 M salt).

Also included are nucleic acids that hybridize under low, moderate, and high stringency conditions, as defined herein, to all or a portion (e.g., the portion encoding the variable region) of the nucleotide sequence represented by isolated polynucleotide sequence(s) that encode an antibody or antibody fragment of the present disclosure. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, at least 98%, or at least 99% identical to the sequence of a portion or all of a nucleic acid encoding an anti-IL-25 polypeptide (e.g., a heavy chain or light chain variable region), or its complement. Hybridizing nucleic acids of the type described herein can be used, for example, as a cloning probe, a primer, e.g., a PCR primer, or a diagnostic probe.

### Polynucleotides, Vectors, and Cells

Other embodiments encompass isolated polynucleotides that comprise a sequence encoding an anti-IL-25 antibody or antibody fragment thereof, vectors, and cells comprising the polynucleotides, and recombinant techniques for production of the antibody. The isolated polynucleotides can encode any desired form of the anti-IL-25 antibody including, for example, full length monoclonal antibodies, Fab, Fab, F(ab)2, and Fv fragments, diabodies, linear antibodies, single-chain antibody molecules, miniantibodies, and multispecific antibodies formed from antibody fragments.

Some embodiments include isolated polynucleotides comprising sequences that encode the heavy chain variable region of an antibody or antibody fragment having the amino acid sequence of SEQ ID NOs: 1, 3, 5, 7, 9, 44 and 47. Some embodiments include isolated polynucleotides comprising sequences that encode the light chain variable region of an antibody or antibody fragment having the amino acid sequence of SEQ ID NOs: 2, 4, 6, 8, 10, 45, 46 and 48.

In an embodiment, the isolated polynucleotide sequence(s) encodes an antibody or antibody fragment having a heavy chain and a light chain variable region comprising the amino acid sequences of:
(a) a variable heavy chain sequence comprising SEQ ID NO: 1 and a variable light chain sequence comprising SEQ ID NO: 2;
(b) a variable heavy chain sequence comprising SEQ ID NO: 3 and a variable light chain sequence comprising SEQ ID NO: 4;
(c) a variable heavy chain sequence comprising SEQ ID NO: 5 and a variable light chain sequence comprising SEQ ID NO: 6,
(d) a variable heavy chain sequence comprising SEQ ID NO: 7 and a variable light chain sequence comprising SEQ ID NO: 8;
(e) a variable heavy chain sequence comprising SEQ ID NO: 9 and a variable light chain sequence comprising SEQ ID NO: 10;
(f) a variable heavy chain sequence comprising SEQ ID NO: 44 and a variable light chain sequence comprising SEQ ID NO: 45,
(g) a variable heavy chain sequence comprising SEQ ID NO: 44 and a variable light chain sequence comprising SEQ ID NO. 46; or
(h) a variable heavy chain sequence comprising SEQ ID NO: 47 and a variable light chain sequence comprising SEQ ID NO: 48.

In another embodiment, the isolated polynucleotide sequence(s) encodes an antibody or antibody fragment having a light chain and a heavy chain variable region comprising the amino acid sequences of:
(a) a variable heavy chain sequence that is 90%, 95%, or 99% identical to SEQ ID NO: 1 and a variable light chain sequence that is 90%, 95%, or 99% identical to SEQ ID NO: 2;
(b) a variable heavy chain sequence that is 90%, 95%, or 99% identical to SEQ ID NO: 3 and a variable light chain sequence that is 90%, 95%, or 99% identical to SEQ ID NO: 4;
(c) a variable heavy chain sequence that is 90%, 95%, or 99% identical to SEQ ID NO: 5 and a variable light chain sequence that is 90%, 95%, or 99% identical to SEQ ID NO: 6;
(d) a variable heavy chain sequence that is 90%, 95%, or 99% identical to SEQ ID NO: 7 and a variable light chain sequence that is 90%, 95%, or 99% identical to SEQ ID NO: 8;
(e) a variable heavy chain sequence that is 90%, 95%, or 99% identical to SEQ ID NO: 9 and a variable light chain sequence that is 90%, 95%, or 99% identical to SEQ ID NO: 10;
(f) a variable heavy chain sequence that is 90%, 95%, or 99% identical to SEQ ID NO: 44 and a variable light chain sequence that is 90%, 95%, or 99% identical to SEQ ID NO: 45;
(g) a variable heavy chain sequence that is 90%, 95%, or 99% identical to SEQ ID NO: 44 and a variable light chain sequence that is 90%, 95%, or 99% identical to SEQ ID NO: 46; or
(h) a variable heavy chain sequence that is 90%, 95%, or 99% identical to SEQ ID NO: 47 and a variable light chain sequence that is 90%, 95%, or 99% identical to SEQ ID NO: 48.

The polynucleotide(s) that comprise a sequence encoding an anti-IL-25 antibody or antibody fragment thereof can be fused to one or more regulatory or control sequence, as known in the art, and can be contained in suitable expression vectors or cells as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the antibody or antibody fragment thereof is inserted into a replicable vector for cloning (amplification of the DNA) or for expression. Many suitable vectors for expressing the recombinant antibody are available. The vector components generally include, but are not limited to, one or more of the following: a signal sequence, an origin of replication, one or more marker genes, an enhancer element, a promoter, and a transcription termination sequence.

The anti-IL-25 antibody or antibody fragment thereof can also be produced as fusion polypeptides, in which the antibody or fragment thereof is fused with a heterologous polypeptide, such as a signal sequence or other polypeptide having a specific cleavage site at the amino terminus of the mature protein or polypeptide. The heterologous signal sequence selected is typically one that is recognized and processed (i.e., cleaved by a signal peptidase) by the cell. For prokaryotic cells that do not recognize and process the anti-IL-25 antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO 90/13646. In mammalian cells, mammalian signal sequences as well as viral secretory leaders, for example, the herpes simplex gD signal, can be used. The DNA for such precursor region is ligated in reading frame to DNA encoding the anti-IL-25 antibody.

Expression and cloning vectors contain a nucleic acid sequence that enables the vector to replicate in one or more selected cells. Generally, in cloning vectors this sequence is one that enables the vector to replicate independently of the host chromosomal DNA and includes origins of replication or autonomously replicating sequences. Such sequences are well known for a variety of bacteria, yeast, and viruses. The origin of replication from the plasmid pBR322 is suitable for most Gram-negative bacteria, the 2-υ. plasmid origin is suitable for yeast, and various viral origins (SV40, polyoma, adenovirus, VSV, and BPV) are useful for cloning vectors in mammalian cells. Generally, the origin of replication component is not needed for mammalian expression vectors (the SV40 origin may typically be used only because it contains the early promoter).

Expression and cloning vectors may contain a gene that encodes a selectable marker to facilitate identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

### Non-Therapeutic Uses

The anti-IL-25 antibody or antibody fragment thereof described herein are useful as affinity purification agents. In this process, the antibodies are immobilized on a solid phase such a Protein A resin, using methods well known in the art. The immobilized antibody is contacted with a sample containing the IL-25 protein (or fragment thereof) to be purified, and thereafter the support is washed with a suitable solvent that will remove substantially all the material in the sample except the IL-25 protein, which is bound to the immobilized antibody. Finally, the support is washed with another suitable solvent that will release the IL-25 protein from the antibody.

An anti-IL-25 antibody or antibody fragment thereof is also useful in diagnostic assays to detect and/or quantify IL-25 protein, for example, detecting IL-25 expression in specific cells, tissues, or serum. The anti-IL-25 antibodies can be used diagnostically to, for example, monitor the development or progression of a disease as part of a clinical testing procedure to, e.g., determine the efficacy of a given treatment and/or prevention regimen. Detection can be facilitated by coupling the anti-IL-25 antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present disclosure.

The anti-IL-25 antibody or antibody fragment thereof can be used in methods for diagnosing an IL-25-associated disorder (e.g., a disorder characterized by abnormal expression of IL-25) or to determine if a subject has an increased risk of developing an IL-25-associated disorder. Such methods include contacting a biological sample from a subject with an anti-IL-25 antibody or antibody fragment thereof and detecting binding of the antibody to IL-25. By “biological sample” is intended any biological sample obtained from an individual, cell line, tissue culture, or other source of cells potentially expressing IL-25. Methods for obtaining tissue biopsies and body fluids from mammals are well known in the art.

In some embodiments, the method can further comprise comparing the level of IL-25 in a patient sample to a control sample (e.g., a subject that does not have an IL-25-associated disorder) to determine if the patient has an IL-25-associated disorder or is at risk of developing an IL-25-associated disorder.

It will be advantageous in some embodiments, for example, for diagnostic purposes to label the antibody with a detectable moiety. Numerous detectable labels are available, including radioisotopes, fluorescent labels, enzyme substrate labels and the like. The label may be indirectly conjugated with the antibody using various known techniques. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody can be conjugated with a small hapten (such as digoxin) and one of the different types of labels mentioned above is conjugated with an anti-hapten antibody (e.g., anti-digoxin antibody). Thus, indirect conjugation of the label with the antibody can be achieved.

Exemplary radioisotopes labels include 35S, 14C, 125I, 3H, and 131I. The antibody can be labeled with the radioisotope, using the techniques described in, for example, Current Protocols in Immunology, Volumes 1 and 2, 1991, Coligen et al., Ed. Wiley-Interscience, New York, N.Y., Pubs. Radioactivity can be measured, for example, by scintillation counting.

Exemplary fluorescent labels include labels derived from rare earth chelates (europium chelates) or fluorescein and its derivatives, rhodamine and its derivatives, dansyl, Lissamine, phycoerythrin, and Texas Red are available. The fluorescent labels can be conjugated to the antibody via known techniques, such as those disclosed in Current Protocols in Immunology, for example. Fluorescence can be quantified using a fluorimeter.

There are various well-characterized enzyme-substrate labels known in the art (see, e.g., U.S. Pat. No. 4,275,149). The enzyme generally catalyzes a chemical alteration of the chromogenic substrate that can be measured using various techniques. For example, alteration may be a color change in a substrate that can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured, using a chemiluminometer, for example, or donates energy to a fluorescent acceptor.

Examples of enzymatic labels include luciferases such as firefly luciferase and bacterial luciferase (U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (such as glucose oxidase, galactose oxidase, and glucose-6-phosphate dehydrogenase), heterocydic oxidases (such as uricase and xanthine oxidase), lactoperoxidase, microperoxidase, and the like. Techniques for conjugating enzymes to antibodies are described, for example, in O'Sullivan et al., 1981, Methods for the Preparation of Enzyme-Antibody Conjugates for use in Enzyme Immunoassay, in Methods in Enzym. (J. Langone & H. Van Vunakis, eds.), Academic press, N.Y., 73: 147-166.

Examples of enzyme-substrate combinations include, for example: Horseradish peroxidase (HRPO) with hydrogen peroxidase as a substrate, wherein the hydrogen peroxidase oxidizes a dye precursor such as orthophenylene diamine (OPD) or 3,3,5,5-tetramethyl benzidine hydrochloride (TMB); alkaline phosphatase (AP) with para-Nitrophenyl phosphate as chromogenic substrate; and β-D-galactosidase (β-D-Gal) with a chromogenic substrate such as p-nitrophenyl-β-D-galactosidase or fluorogenic substrate 4-methylumbelliferyl-β-D-galactosidase.

In another embodiment, the anti-IL-25 antibody or antibody fragment thereof is used unlabeled and detected with a labeled antibody that binds the anti-IL-25 antibody or antibody fragment thereof.

The antibodies and antibody fragments thereof described herein may be employed in any known assay method, such as competitive binding assays, direct and indirect sandwich assays, and immunoprecipitation assays. See, e.g., Zola, Monoclonal Antibodies: A Manual of Techniques, pp. 147-158 (CRC Press, Inc. 1987).

The anti-IL-25 antibody or antibody fragment thereof can be used to inhibit the binding of ligand to the IL-25 receptor. Such methods comprise administering an anti-IL-25 antibody to a cell (e.g., a mammalian cell) or cellular environment, whereby signaling mediated by the IL-25 receptor is inhibited. These methods can be performed in vitro or in vivo. By “cellular environment” is intended the tissue, medium, or extracellular matrix surrounding a cell.

### Compositions and Methods of Treatment

The disclosure also provides compositions including, for example, pharmaceutical compositions that comprise an anti-IL-25 antibody or antibody fragment thereof. Such compositions have numerous therapeutic uses for the treatment, prevention, or amelioration of diseases or disorders (e.g., diseases or disorders involving a biological activity mediated by the IL-25/IL-25 receptor signaling axis) such as an immune-mediated inflammatory disorder or an autoimmune disease.

An anti-IL-25 antibody or antibody fragment thereof disclosed herein is useful in the treatment of various diseases or disorders, including Type 2 inflammation diseases, autoimmune diseases, or cancer. In particular, the disclosed antibodies are useful for the treatment of immune-mediated airway inflammatory disorders.

Methods for treating an IL-25 associated disorder comprise administering a therapeutically effective amount of an anti-IL-25 antibody or antibody fragment thereof to a subject in need thereof. The present disclosure also provides methods for the treatment or prevention of a Type 2 inflammatory disease comprising administering a composition or formulation that comprises an anti-IL-25 antibody or antibody fragment thereof, and optionally another immune-based therapy, to a subject in need thereof. The disclosed anti-IL-25 antibodies may be administered as an add-on therapy or in combination with other cytokine inhibitors.

The disclosed antibodies are also useful in methods of preventing or treating of airway hyperresponsiveness (AHR) or airway inflammation, including but not limited to allergic asthma, non-allergic asthma, severe refractory asthma, asthma exacerbations, viral-induced asthma, viral-induced asthma exacerbations, steroid-resistant asthma, steroid-sensitive asthma, eosinophilic asthma, non-eosinophilic asthma, and related disorders. Additionally, the term includes viral-induced asthma exacerbations.

The disclosed antibodies are also useful in methods of preventing or treating chronic obstructive pulmonary disease (COPD) in a patient in need thereof by administering an anti-IL-25 antibody or antigen-binding fragment thereof as described herein.

The disclosed antibodies are also useful in methods of preventing or treating inflammatory bowel disease (IBD). In this context, “IBD” refers to a group of disorders that affect the large intestine or colon's mucosal layer and include ulcerative colitis, Crohn's disease, collagenous colitis, lymphocytic colitis, ischemic colitis, diversion colitis, Behcet's syndrome, infective colitis, indeterminate colitis, and other related conditions characterized by inflammation.

The disclosed antibodies are also useful in methods of preventing or treating atopic dermatitis (AD), including but not limited to inflammatory skin conditions marked by severe itching and eczematous lesions that are scaly and dry. AD may result from epidermal barrier dysfunction, allergy to certain foods, pollen, mold, dust mite, animals radiation exposure, and/or asthma. The methods disclosed herein can be utilized to treat various degrees of AD, including mild, moderate, moderate-to-severe, and severe forms.

The disclosed antibodies are also useful in methods of preventing or treating various diseases and disorders such as Eosinophilic Granulomatosis with Polyangiitis or EGPA (also known as Churg-Strauss Syndrome), allergy, allergic rhinitis, allergic airway inflammation, food hypersensitivity, urticaria (including chronic idiopathic urticaria) eosinophilic pneumonia, eosinophilic esophagitis, hypereosinophilic syndrome, idiopathic pulmonary fibrosis, hypersensitivity pneumonitis, rheumatoid arthritis, vasculitis, uveitis, cancer, and graft-versus-host-disease.

The disclosed antibodies are also useful in methods of treatment of cancer, either alone (e.g., as monotherapies) or in combination with other immunotherapeutic agents and/or a chemotherapy.

The antibodies can be administered either alone or in combination with other compositions that are useful for treating an immune-mediated inflammatory disorder or an autoimmune disease. In some embodiments, compositions including, for example, pharmaceutical compositions, comprising the anti-IL-25 antibody can further comprise a therapeutic agent, either conjugated or unconjugated to the binding agent.

In some aspects, a composition, e.g., a pharmaceutical composition is provided that comprises one or more antibodies disclosed herein. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, bispecific and multi-specific molecule, may be coated in a material to protect the compound from the action of acids and other natural conditions that may inactivate the compound.

A composition can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978.

Dosage levels of the active ingredients in the pharmaceutical compositions may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The pharmaceutical compositions described herein may be administered in effective amounts. An “effective amount” refers to the amount which achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease.

In some aspects, the compositions described herein are administered to patients, e.g., in vivo, to treat or prevent a variety of disorders such as those described herein. Preferred patients include human patients having disorders that can be corrected or ameliorated by administering the agents modulate a biological activity of the IL-25/IL-25 receptor signaling axis.

In some aspects, conventional viral and non-viral based gene transfer methods can be used to introduce nucleic acids encoding the antibodies or derivatives thereof, as described herein, in mammalian cells or target tissues. Such methods can be used to administer nucleic acids encoding the antibodies to cells in vitro. In some embodiments, the nucleic acids encoding the antibodies or derivatives thereof are administered for in vivo or ex vivo gene therapy uses. In other embodiments, gene delivery techniques are used to study the activity of the antibodies in cell based or animal models. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Such methods are well known in the art.

Methods of non-viral delivery of nucleic acids encoding engineered polypeptides of the disclosure include lipofection, microinjection, biolistics, virosomes, liposomes, immunoliposomes, polycation or lipid:nucleic acid conjugates, naked DNA, artificial virions, and agent-enhanced uptake of DNA. Lipofection methods and lipofection reagents are well known in the art (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration). The preparation of lipid:nucleic acid complexes, including targeted liposomes such as immunolipid complexes, is well known to one of skill in the art.

The use of RNA or DNA viral based systems for the delivery of nucleic acids encoding the antibodies described herein take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the disclosure could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

All patents and publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The broad scope of this disclosure is best understood with reference to the following examples, which are not intended to limit the disclosures to the specific embodiments. The specific embodiments described herein are offered by way of example only, and the disclosure is to be limited by the terms of the appended claims, along with the full scope of the equivalents to which such claims are entitled.

### Illustration of Subject Technology as Clauses

Various examples of aspects are described as numbered clauses (1, 2, 3, etc.) for convenience. These are provided as examples, and do not limit the subject technology. Identifications of the reference numbers are provided below merely as examples and for illustrative purposes, and the clauses are not limited by those identifications.

Clause 1: an anti-IL-25 antibody comprising: (a) a heavy chain variable region comprising the amino acid sequence of SEQ ID NO: 23 (HCDR1); the amino acid sequence of SEQ ID NO: 49 (HCDR2); and the amino acid sequence of SEQ ID NO: 25 (HCDR3); and a light chain variable region comprising the amino acid sequence of SEQ ID NO: 50 (LCDR1); the amino acid sequence of SEQ ID NO: 51 (LCDR2); and the amino acid sequence of SEQ ID NO: 28 (LCDR3); (b) a heavy chain variable region comprising the amino acid sequence of SEQ ID NO: 23 (HCDR1); the amino acid sequence of SEQ ID NO: 52 (HCDR2); and the amino acid sequence of SEQ ID NO: 25 (HCDR3); and a light chain variable region comprising the amino acid sequence of SEQ ID NO: 50 (LCDR1); the amino acid sequence of SEQ ID NO: 51 (LCDR2); and the amino acid sequence of SEQ ID NO: 28 (LCDR3); (c) a heavy chain variable region comprising the amino acid sequence of SEQ ID NO: 11 (HCDR1); the amino acid sequence of SEQ ID NO: 12 (HCDR2); and the amino acid sequence of SEQ ID NO: 13 (HCDR3); and a light chain variable region comprising the amino acid sequence of SEQ ID NO: 14 (LCDR1); the amino acid sequence of SEQ ID NO: 15 (LCDR2), and the amino acid sequence of SEQ ID NO: 16 (LCDR3); (d) a heavy chain variable region comprising the amino acid sequence of SEQ ID NO: 17 (HCDR1); the amino acid sequence of SEQ ID NO: 18 (HCDR2); and the amino acid sequence of SEQ ID NO: 19 (HCDR3); and a light chain variable region comprising the amino acid sequence of SEQ ID NO: 20 (LCDR1); the amino acid sequence of SEQ ID NO: 21 (LCDR2); and the amino acid sequence of SEQ ID NO: 22 (LCDR3), (e) a heavy chain variable region comprising the amino acid sequence of SEQ ID NO: 23 (HCDR1); the amino acid sequence of SEQ ID NO: 24 (HCDR2); and the amino acid sequence of SEQ ID NO: 25 (HCDR3); and a light chain variable region comprising the amino acid sequence of SEQ ID NO: 26 (LCDR1); the amino acid sequence of SEQ ID NO: 27 (LCDR2); and the amino acid sequence of SEQ ID NO: 28 (LCDR3); (f) a heavy chain variable region comprising the amino acid sequence of SEQ ID NO 29 (HCDR1); the amino acid sequence of SEQ ID NO: 30 (HCDR2); and the amino acid sequence of SEQ ID NO: 31 (HCDR3); and a light chain variable region comprising the amino acid sequence of SEQ ID NO: 32 (LCDR1); the amino acid sequence of SEQ ID NO: 33 (LCDR2); and the amino acid sequence of SEQ ID NO: 34 (LCDR3); or (g) a heavy chain variable region comprising the amino acid sequence of SEQ ID NO: 35 (HCDR1); the amino acid sequence of SEQ ID NO: 36 (HCDR2); and the amino acid sequence of SEQ ID NO: 37 (HCDR3); and a light chain variable region comprising the amino acid sequence of SEQ ID NO: 38 (LCDR1); the amino acid sequence of SEQ ID NO: 39 (LCDR2); and the amino acid sequence of SEQ ID NO: 40 (LCDR3).

Clause 2: the anti-IL-25 antibody or an antigen-binding fragment thereof of clause 1, wherein the antibody or antigen-binding fragment thereof comprises a heavy chain variable region comprising the amino acid sequence of any one of SEQ ID NOs: 1, 3, 5, 7, 9, 44 or 47; and a light chain variable region comprising the amino acid sequence of any one of SEQ ID NOs: 2, 4, 6, 8, 10, 45, 46 or 48.

Clause 3: the anti-IL-25 antibody of clause 1, wherein the antibody comprises: (a) a heavy chain variable region sequence of SEQ ID NO: 44 and a light chain variable region sequence of SEQ ID NO: 45; (b) a heavy chain variable region sequence of SEQ ID NO: 44 and a light chain variable region sequence of SEQ ID NO: 46; (c) a heavy chain variable region sequence of SEQ ID NO: 47 and a light chain variable region sequence of SEQ ID NO: 48; (d) a heavy chain variable region sequence of SEQ ID NO: 1 and a light chain variable region sequence of SEQ ID NO: 2; (e) a heavy chain variable region sequence of SEQ ID NO: 3 and a light chain variable region sequence of SEQ ID NO: 4; (f) a heavy chain variable region sequence of SEQ ID NO: 5 and a light chain variable region sequence of SEQ ID NO: 6; (g) a heavy chain variable region sequence of SEQ ID NO: 7 and a light chain variable region sequence of SEQ ID NO: 8; or (h) a heavy chain variable region sequence of SEQ ID NO: 9 and a light chain variable region sequence of SEQ ID NO: 10.

Clause 4: the anti-IL-25 antibody of clause 1, wherein the antibody is an anti-human anti-IL-25 antibody.

Clause 5: the anti-IL-25 antibody of clause 1, wherein the antibody is a full-length antibody comprising a human IgG1 constant region selected from SEQ ID NO: 55 or SEQ ID NO: 56.

Clause 6: the anti-IL-25 antibody of clause 1, wherein the antibody is an antibody fragment.

Clause 7: the anti-IL-25 antibody of clause 4, wherein the antibody fragment is selected from the group consisting of: Fab, Fab, F(ab)2, Fd, Fv, scFv and scFv-Fc fragment, a single-chain antibody, a minibody, and a diabody.

Clause 8: the anti-IL-25 antibody of clause 1, wherein the antibody is a monoclonal antibody.

Clause 9: the anti-IL-25 antibody of clause 1, wherein the antibody is a human antibody.

Clause 10: the anti-IL-25 antibody of clause 1, wherein the antibody is a murine antibody.

Clause 11: the anti-IL-25 antibody of clause 1, wherein the antibody is a chimeric antibody.

Clause 12: the anti-IL-25 antibody of clause 1, wherein the antibody is a bispecific or a multi-specific antibody.

Clause 13: the anti-IL-25 antibody of clause 1, wherein the antibody is a humanized antibody.

Clause 14: a pharmaceutical composition comprising the antibody of clause 1 and a pharmaceutically acceptable carrier.

Clause 15: a method of treating and/or preventing a Type 2 inflammation disease, autoimmune disease, allergic disorder, or cancer in a subject in need thereof, the method comprising: administering to the subject the antibody of clause 1.

Clause 16: a composition of polynucleotides comprising a) a first polynucleotide coding for a heavy chain variable region comprising the amino acid sequence set forth in SEQ ID NO: 1, 3, 5, 7, 9, 44, or 47; and b) a second polynucleotide coding for a light chain variable region comprising the amino acid sequence set forth in SEQ ID NO: 2, 4, 6, 8, 10, 45, 46, or 48.

Clause 17: A composition of vectors comprising: a) a first vector comprising the first polynucleotide of clause 16; and b) a second vector comprising the second polynucleotide of clause 16.

Clause 18: a cell comprising the polynucleotide composition according to clause 16, or a vector composition according to clause 17.

Clause 19: a method for the production of an anti-IL-25 antibody according to clause 1, the method comprising culturing the cell of clause 18 in a culture media; and recovering the anti-IL-25 antibody from the media.

### EXAMPLES

### General Methods

Methods for protein purification including immunoprecipitation, chromatography, and electrophoresis are described. See, e.g., Coligan et al. (2000) Current Protocols in Protein Science, Vol. 1, John Wiley and Sons, Inc., New York. Chemical analysis, chemical modification, post-translational modification, production of fusion proteins, and glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra.

Hybridoma or cell culture supernatant containing an anti-TL-25 antibody was purified via a HiTrap protein G column (GE, cat. No. 17040401) according to the manufacturer's protocol. Briefly, the column was equilibrated with DPBS (Gibco, cat. No. 14190-136) for 5 CV and supernatant was loaded via syringe/infusion pump (Legato 200, KDS) at ambient temperature and a 3 minute residence time. The column was washed with 5 CV of DPBS and elution was performed with 4 CV of pH 2.8 elution buffer (Fisher Scientific, cat. No. PI21004), neutralized with 1M Tris-HCL, pH 8.5 (Fisher Scientific, cat No. 50-843-270) and assayed by A280 (DropSense96, Trinean). The purified material was then buffer exchanged into DPBS via 30 kDa MWCO centrifugal filters (EMD Millipore, cat. No. UFC803024). The final pool was analyzed by A280 and stored at either 2-8° C. or −20° C.

Standard methods in molecular biology are described. See, e.g., Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

The sequences for the heavy and light chain variable regions for hybridoma clones were determined as described below. Total RNA was extracted from 1-2×10⁶ hybridoma cells using the RNeasy Plus Mini Kit from Qiagen (Germantown, MD, USA). CDNA was generated by performing 5′ RACE reactions using the SMARTer RACE 5′/3′ Kit from Takara (Mountainview, CA, USA). PCR was performed using the Q5 High-Fidelity DNA Polymerase from NEB (Ipswich, MA, USA) to amplify the variable regions from the heavy and light chains using the Takara Universal Primer Mix in combination with gene specific primers for the 3′ mouse constant region of the appropriate immunoglobulin. The amplified variable regions for the heavy and light chains were run on 2% agarose gels, the appropriate bands excised and then gel purified using the Mini Elute Gel Extraction Kit from Qiagen. The purified PCR products were cloned using the Zero Blunt PCR Cloning Kit from Invitrogen (Carlsbad, CA, USA), transformed into Stellar Competent E Coli cells from Takara and plated onto LB Agar+50 μg/ml kanamycin plates. Direct colony Sanger sequencing was performed by GeneWiz (South Plainfield, NJ, USA). The resulting nucleotide sequences were analyzed using IMGT V-QUEST to identify productive rearrangements and analyze translated protein sequences. CDR determination was based on Kabat numbering.

Selected VH or VL chains were PCR amplified and cloned into a pcDNA3.4-based expression vector, which harbors the constant region from human IgG1 (Uniprot P01857 or its allotype) or human Kappa light chain (UniProt P01834). Paired heavy chain- and light chain-expressing plasmids were transfected into Expi293 cells (Thermo Fisher Scientific) following provider's Expi293 expression system protocol. Five days after transfection culture supernatants were collected by centrifugation. Chimera antibodies were purified by 1-step affinity purification using Protein A column and buffer exchanged to PBS pH 7.2 or 20 mM Histidine pH 5.5.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard techniques for characterizing ligand/receptor interactions are available. See, e.g., Coligan et al. (2001) Current Protocols in Immunology, Vol. 4, John Wiley, Inc., New York. Standard methods of antibody functional characterization appropriate for the characterization of antibodies with particular mechanisms of action are also well known to those of skill in the art.

Two in-house IL-25-specific antibodies referred to herein as IL-25-PC2 and IL-25-PC4 were prepared. IL-25-PC2 was prepared based on the publicly available information published in WO 2016/049000 A2 (VH, SEQ ID NO: 114; and VL, SEQ ID NO: 122) and IL-25-PC4 was prepared based on the publicly available information published in WO 2020/102935 A1 (VH, SEQ TD NO: 12; and VL, SEQ ID NO: 13). Both antibodies were used to establish the binding and functional assays used to evaluate and characterize the anti-IL-25 specific antibodies disclosed herein.

Software packages and databases for determining, e.g., antigenic fragments, leader sequences, protein folding, functional domains, CDR annotation, glycosylation sites, and sequence alignments, are available.

### Example 1: Generation of Anti-IL-25 Antibodies

Anti-IL-25 antibodies were generated by immunizing wild-type mice or transgenic/humanized mice.

Immunization. Mice were immunized with recombinant human IL-25 protein, either intraperitoneally, subcutaneously, footpad or base of tail. The immune response was monitored by retroorbital bleeds. The plasma was screened by ELISA (as described below), and mice with sufficient titers of anti-human TL-25 were used for fusions. Mice were boosted intraperitoneally, intravenously or footpad or based of tail with recombinant human IL-25 protein before sacrifice and removal of the spleen and lymph nodes.

Selection of Balb/c Mice Producing Anti-IL-25 Antibodies. To select Balb/c mice that producing antibodies that bound IL-25, sera from immunized mice were screened by ELISA for binding to IL-25. Briefly, recombinant human IL-25 protein coated ELISA plate was incubated with dilutions of serum from immunized mice for one hour at room temperature, plate was washed, and specific antibody binding was detected with HRP-labeled anti-mouse IgG antibody. Plate was read using an ELISA reader machine (Biotek). Hybridomas supernatants were tested for anti-IL-25 specific binding by ELISA as described above.

Generation of Hybridomas Producing Antibodies to IL-25. To generate hybridomas producing antibodies of the invention, splenocytes and lymph node cells were isolated from an immunized mouse fused to an appropriate immortalized cell line, such as a mouse myeloma cell line. The resulting hybridomas were screened for the production of antigen-specific antibodies. For example, single cell suspensions of splenocytes, lymph node cells from immunized mice were fused to equal number of Sp2/0 non-secreting mouse IgG myeloma cells (ATCC, CRL 1581) by electrofusion. Cells were plated in flat bottom 96-well tissue culture plates, followed by 2 weeks of incubation in selection medium (HAT medium), then switched to hybridoma culture media. Approximately 10-14 days after cell plating, supernatants from individual wells were screened by ELISA as described above. The antibody secreting hybridomas were transferred to 24-well plates, screened again, and if still positive for anti-IL-25, the positive hybridomas were subcloned by limiting dilution or sorting using a single cell sorter. The stable subclones were then cultured in vitro to generate small amounts of antibodies to be used for purification and for characterization.

### Example 2: Binding of Anti-IL-25 Specific Antibodies

In this study, the ability of five disclosed anti-IL-25 antibodies (IL25Ab1 to Ab5) binding to both human and mouse IL-25 was evaluated using ELISA. Briefly, recombinant human IL-25 protein (Acro, catalog no: IL5-H4221, lot no: 401-20CNF1-UT) or recombinant mouse IL-25 protein (Sino Biological Inc, catalog no: 50138-M07H, lot no: LC15AP0607) was directly coated to ELISA plates, respectively. Recombinant antibodies were then added to the plates followed by detection using goat-anti-human IgG-HRP (Jackson ImmunoResearch, catalog no: 109-035-098, Lot no: 157400). After the addition of ABTS (Moss Inc., catalog no: ABTS-1000, Lot no: 03086202) substrate, ELISA plates were read using an ELISA plate reader (Bioteck).

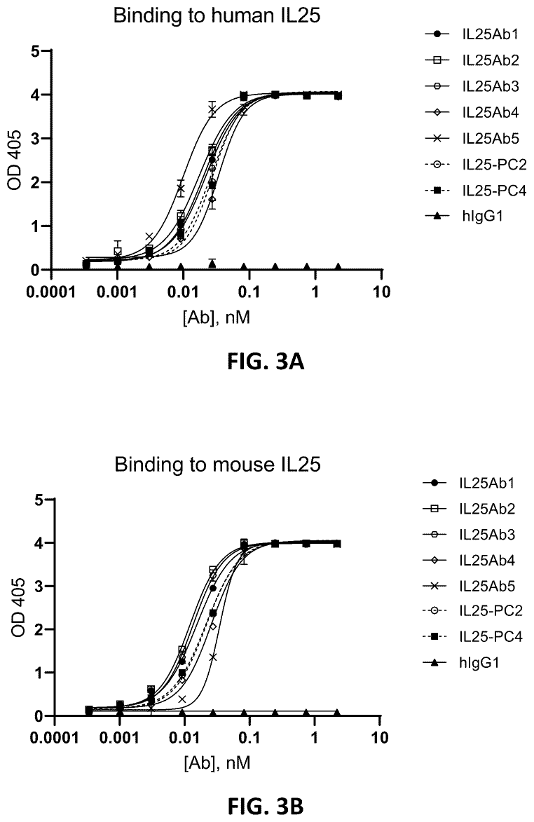

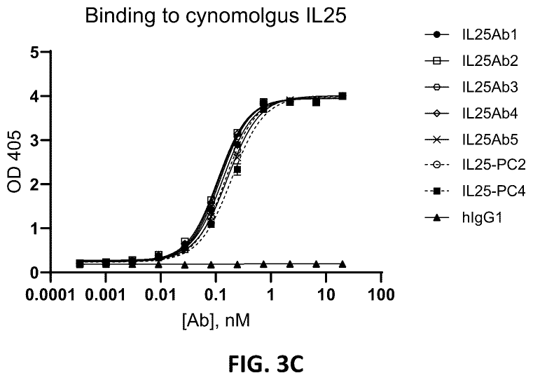

FIG. 3A shows that the five disclosed anti-IL-25 antibodies bound to the human recombinant IL-25 protein in a dose-dependent manner, with EC50 values ranging from 0.010 nM to 0.034 nM. The positive control antibodies IL-25-PC2 (A recombinant antibody made by NovaRock, Lot no: P09282021JLF) and IL-25-PC4 (A recombinant antibody made by NovaRock, Lot no: A10032022JLF) had EC₅₀ values of 0.026 nM and 0.028 nM, respectively. The human IgG1 (InVivoMab, catalog no: BE0297, Lot no: 760620M1) isotype control did not show any binding.

FIG. 3B shows that the five disclosed anti-IL-25 antibodies also bound to the mouse recombinant IL-25 protein in a dose-dependent manner, with EC₅₀ values ranging from 0.012 nM to 0.035 nM. The positive control antibodies IL-25-PC2 and IL-25-PC4 had EC50 values of 0.022 nM and 0.021 nM, respectively.

In another study, the ability of five disclosed anti-IL-25 antibodies (IL25Ab1 to Ab5) binding to cynomolgus IL-25 was evaluated using ELISA. Briefly, recombinant human IL-25 protein (Acro, catalog no: IL5-H4221, lot no: 401-20CNF1-UT) or recombinant cynomolgus IL-25 protein (Sino Biological Inc, catalog no: customized order, lot no: MB17MA2715) was directly coated to ELISA plates, respectively. Recombinant antibodies were then added to the plates followed by detection using goat-anti-human Kappa Light Chain antibody HRP (Novus, catalog no: NBP1-75064, Lot no: 68-188-010920). After the addition of ABTS (Moss Inc., catalog no: ABTS-1000, Lot no: 03086202) substrate, ELISA plates were read using an ELISA plate reader (Bioteck).

FIG. 3C shows that the five disclosed anti-IL-25 antibodies also bound to the cynomolgus recombinant IL-25 protein in a dose-dependent manner, with similar binding activity when compared to the positive control antibodies IL-25-PC2 and IL-25-PC4.

TABLE 5
Binding EC₅₀ of the IL-25 Antibodies to IL-25 proteins

Goat-anti-human IgG-HRP

| | Human IL-25 | Mouse IL-25 |
|---|---|---|
| Detection | ELISA | ELISA |
| antibody | EC₅₀, nM | EC₅₀, nM |
| IL25Ab1 | 0.020 | 0.015 |
| IL25Ab2 | 0.017 | 0.012 |
| IL25Ab3 | 0.022 | 0.013 |
| IL25Ab4 | 0.034 | 0.026 |
| IL25Ab5 | 0.010 | 0.035 |
| IL25-PC2 | 0.026 | 0.022 |
| IL25-PC4 | 0.028 | 0.021 |

### Example 3: Binding Kinetics of Anti-IL-25 Specific Antibodies

The binding kinetics of the disclosed anti-IL-25 antibodies to recombinant human IL-25 was determined by Octet® Bio-Layer Interferometry (BLI) systems by Sartorius. The assay uses anti-IL-25 monoclonal antibodies as the ligand and recombinant human IL-25 protein as the analyte.

Briefly, the assays were performed by first capturing 5 μg/ml of anti-IL-25 antibodies using anti-human Fc Octet biosensors. The mAb-captured biosensors were then submerged in wells containing serially diluted human IL-25 for 4-6 minutes, followed by a 10-15 minute dissociation time. The binding sensorgrams were collected and analyzed by the Octet Data Analysis software.

Table 6 summarizes the KD values for the anti-IL-25 antibodies disclosed in this study. The data suggests that these antibodies have a strong binding affinity to human recombinant IL-25 and exhibit fast-on and slow-off kinetic characteristics.

TABLE 6
Anti-IL-25 Antibody Binding Kinetics

| Antibody | ka (1/Ms) | kd (1/s) | KD (M) |
|---|---|---|---|
| IL25Ab1 | 1.25E+06 | <1.0E−07 | <1.0E−12 |
| IL25Ab2 | 1.36E+06 | <1.0E−07 | <1.0E−12 |
| IL25Ab3 | 8.75E+05 | <1.0E−07 | <1.0E−12 |
| IL25Ab4 | 1.20E+06 | <1.0E−07 | <1.0E−12 |
| IL25Ab5 | 2.48E+06 | <1.0E−07 | <1.0E−12 |
| IL25-PC2 | 7.80E+05 | <1.0E−07 | <1.0E−12 |

### Example 4: Blocking of IL-25 Induced NF-kB Signaling

IL-25 interacts and signals through an IL-25 receptor comprising the heterodimeric IL-17RA and IL-17RB subunits. The activated heterodimeric receptor recruits the Act1 adaptor, leading to the ubiquitination of TNF receptor-associated factor 6 (TRAF6). This, in turn, triggers a signaling cascade resulting in the activation of NF-κB and AP-1.

A HEK-293 reporter cell line was used to study the blocking activity of the anti-IL-25 antibodies. HEK-Blue™ IL-17 cells (InvivoGen, San Diego, CA) were generated by stable transfection of the human genes encoding the IL-17RA/IL-17RC heterodimeric receptor and Act1 adaptor molecule into the human embryonic kidney HEK293 cell line. These cells also express a secreted embryonic alkaline phosphatase (SEAP) reporter gene that is inducible by NF-κB and AP-1.

In this study, HEK-Blue™ IL-17 cells were treated with recombinant human IL-25 at 5 ng/ml together with serially diluted IL-25 antibodies. After overnight incubation, the NF-κB response was determined using QUANTI-Blue™ Solution, a SEAP detection reagent, by reading the optical density (OD) at 655 nm.

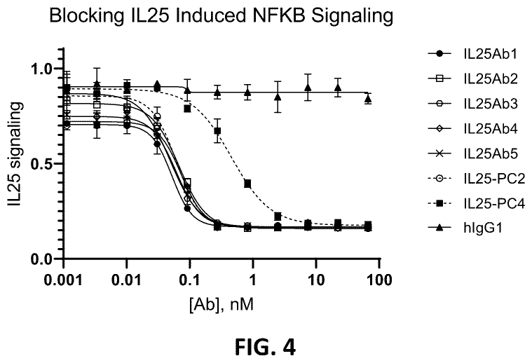

As shown in FIG. 4 and summarized in Table 7, the five disclosed anti-IL-25 antibodies effectively blocked IL-25 induced NF-kB signaling in a dose-dependent manner, with IC₅₀ values ranging from 0.049 nM to 0.073 nM. The positive control antibodies IL-25-PC2 and IL-25-PC4 had IC₅₀ values of 0.062 nM and 0.456 nM, respectively. The human IgG1 isotype control did not show any blocking activity.

TABLE 7
Inhibitory Activity of IL-25 Antibodies in NFKB Signaling Assay

| | IC₅₀, nM |
|---|---|
| IL25Ab1 | 0.052 |
| IL25Ab2 | 0.068 |
| IL25Ab3 | 0.049 |
| IL25Ab4 | 0.073 |
| IL25Ab5 | 0.061 |
| IL25-PC2 | 0.062 |
| IL25-PC4 | 0.456 |

### Example 5: Blocking of IL-25 Induced CXCL1 Production

The expression of interleukin-17 (IL-17) is significantly elevated in the peripheral blood of patients with inflammatory bowel disease (IBD), indicating that IL-17 may play a crucial role in the disease's physiological and pathological processes. Since intestinal epithelial cell line HT-29 has normal colonic epithelial structures and functions, it has been the most common cell line used in laboratory to study the immunologic mechanisms of the intestinal mucosa.

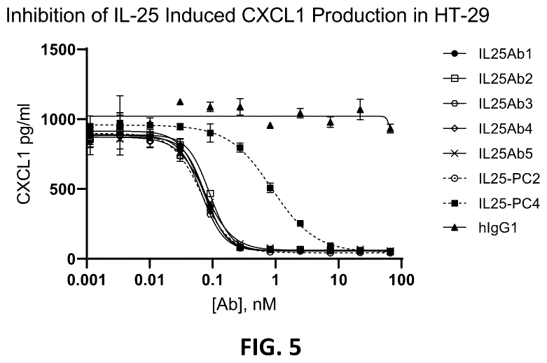

To determine whether the disclosed anti-IL-25 antibodies can block the production of the inflammatory cytokine CXCL1 prompted by human IL-25, HT-29 cells were seeded into assay plates. Subsequently, a combination of serially diluted anti-IL-25 antibody and hIL-25 was added to their respective wells, and the cells were incubated for 72 hours at 37° C. The supernatants were then collected for CXCL1 ELISA, using the Human CXCL1 ELISA Ready-SET-Go kit (R&D system #DY275). The results, which are presented in FIG. 5 and Table 8, demonstrate that five disclosed anti-IL-25 antibodies effectively blocked the production of CXCL1 stimulated by IL-25, thus potentially reducing recruitment of immune cells and preventing the development of inflammation.

The IC₅₀ values ranged from 0.067 nM to 0.089 nM. The positive control antibodies IL-25-PC2 and IL-25-PC4 had IC₅₀ values of 0.066 nM and 0.877 nM, respectively. The human IgG1 isotype control did not show any blocking activity.

TABLE 8
Activity of IL-25 Antibodies in Blocking CXCL1 Production

| | IC₅₀, nM |
|---|---|
| IL25Ab1 | 0.071 |
| IL25Ab2 | 0.089 |
| IL25Ab3 | 0.067 |
| IL25Ab4 | 0.076 |
| IL25Ab5 | 0.077 |
| IL25-PC2 | 0.066 |
| IL25-PC4 | 0.877 |

### Example 6: Blocking of IL-25 Induced IL-5 Production

IL-25 has been implicated as a type 2 cytokine produced by Th2 cells, which was capable of inducing IL-4, IL-5 and IL-13 gene expression. The induction of these cytokines resulted in Th2-like responses marked by increased serum IgE, IgG, and IgA levels, blood eosinophilia, and pathological changes in the lungs and digestive tract that included eosinophilic infiltrates, increased mucus production, and epithelial cell hyperplasia/hypertrophy.

To further characterize the biologic functions of IL-25 antibodies and evaluate their potential in treating inflammatory disease, human PBMCs were used in this assay. When treating the human PBMC with 30 U/ml recombinant hIL-2 and 2 ng/ml recombinant hIL-25 for 6 days, significant amount of IL-5 was produced. However, if the cells were co-treated with IL-25 blocking antibodies, the level of IL-5 was much reduced, suggesting an IL-25 antibody can suppress the type 2 response.

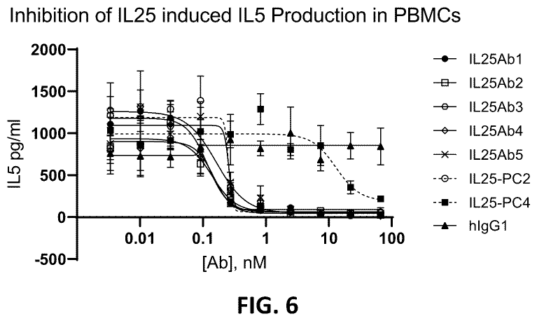

As shown in FIG. 6 and summarized in Table 9, all five disclosed IL-25 antibodies effectively blocked IL-25 induced IL-5 production in a dose-dependent manner, with IC₅₀ values ranging from 0.12 nM to 0.26 nM. The positive control antibodies IL-25-PC2 and IL-25-PC4 had IC₅₀ values of 0.26 nM and 12.99 nM, respectively. The human IgG1 isotype control did not show any blocking activity.

TABLE 9
Activity of IL-25 Antibodies in Blocking IL-5 Production

| | IC₅₀, nM |
|---|---|
| IL25Ab1 | 0.12 |
| IL25Ab2 | 0.14 |
| IL25Ab3 | 0.16 |
| IL25Ab4 | 0.15 |
| IL25Ab5 | 0.26 |
| IL25-PC2 | 0.26 |
| IL25-PC4 | 12.99 |

### Example 7: In Vivo Efficacy of Anti-IL-25 Specific Antibody in OVA-Induced Asthma Model

OVA-induced asthma model was used to study in vivo efficacy of anti-IL-25 specific antibodies. Six to seven-week-old male BALB/c mice were divided into 6 groups (10 mice in each group): (1) control group; (2) OVA group+Vehicle; (3-6) OVA group+anti-IL-25 antibody. Mice in the OVA group were sensitized by intraperitoneal injection of OVA solution containing aluminum hydroxide on day 1 and 14, and challenged by aerosolizing of 1% OVA solution for 30 minutes using BUXCO aerosolizing dosing system on day 28, 29, and 31. Anti-IL-25 antibodies were injected subcutaneously at 10 mg/kg on day 14, 17, 20, 23, 26, 29 and 31. All the tested antibodies were made in mouse IgG1 format. On day 31, pulmonary resistance in response to a range of aerosolized methacholine was measured by whole body plethysmography (WBP, Buxco BFE0100 WBP). On day 32, mice were anesthetized, and the BALF was obtained by flushing the lung with PBS containing 10% FBS.

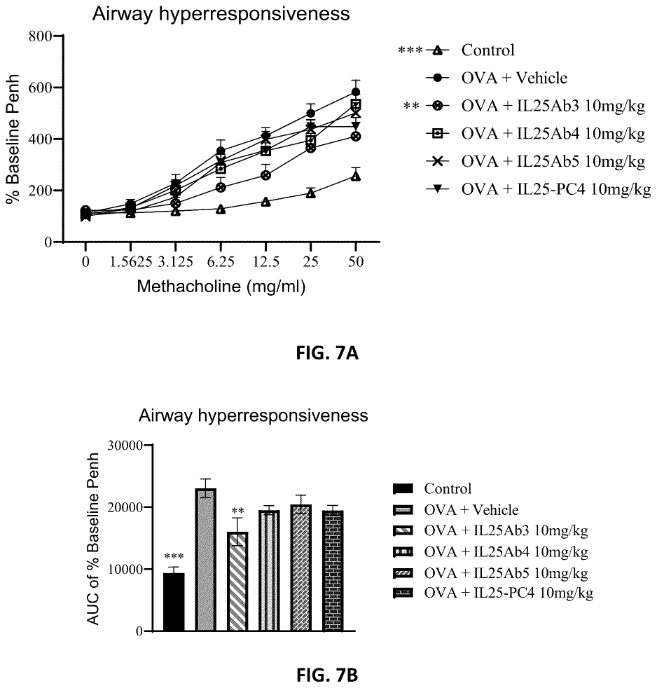

Airway hyperresponsiveness is a hallmark of asthma. In FIG. 7, mice in the OVA+Vehicle group showed significantly enhanced airway resistance to methacholine compared to control group. IL25Ab3 treatment significantly reduced airway resistance. IL25Ab4, IL25Ab5 and IL25-PC4 showed a slight reduction in the airway resistance. However, such reduction did not reach statistically significant. The airway resistance was represented as dose-response data of methacholine presented as percentage change from the baseline level of lung resistance (Penh value) (FIG. 7A) and the AUC of % of baseline Penh (FIG. 7B).

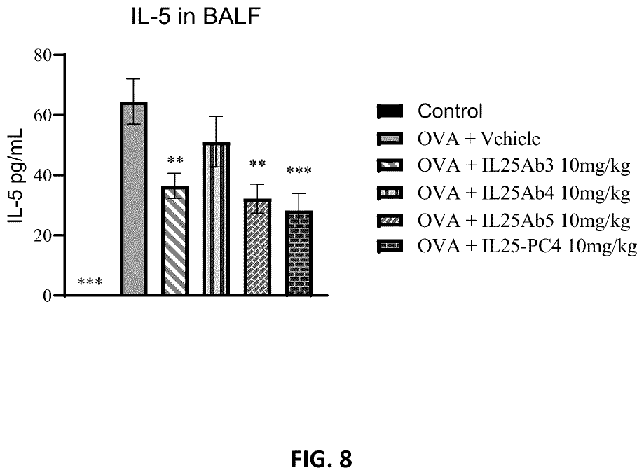

Interleukin-5 (TL-5) exerts a central pathogenic role in differentiation, recruitment, survival, and degranulation of eosinophils. The powerful actions of IL-5 to the induction, maintenance, and amplification of eosinophilic inflammation such as asthma have been reported. In FIG. 8, significant increase of IL-5 in the Bronchoalveolar Lavage Fluid (BALF) in the OVA-induced asthma model when comparing OVA+Vehicle mice to control mice. IL25Ab3, IL25Ab5 and IL25-PC4 treatments significantly reduced production of IL-5 in the BALF. IL25Ab4 treatment decreased the production of IL-5 (not significant).

### Example 8: Humanization of Anti-IL-25Ab3

Murine anti-IL-25Ab3 was humanized using CDR grafting approach. The variable regions of the heavy chain and light chain of murine Ab3 were derived from mouse germline IGHV1-18*01 and IGKV10-94*01 respectively. The heavy chain variable region was used to search against human antibody germline database, and the human germline sequence with the highest similarity, human IGHV1-2*02, was selected as the templates for heavy chain CDR grafting. Similarly, human IGKV1-33*01 was selected as the template for light chain CDR grafting. During grating, structural models of the antibodies were generated, and selected back mutations were introduced to maintain the proper antibody folding. A total of 5 heavy chain variants and 4 light chain variants were designed and cloned into expression constructs. 20 pairs of the heavy chain and light chain humanization variants were made as both Fab and human IgG1 antibody. The Fab fragments of the humanization variants were used to measure binding affinity on BLI instrument, and the human IgG1 antibodies were used for ELISA binding as well as functional assays. Table 10 shows that the selected top 3 humanized Fabs have similar binding affinity as the murine parental Fab.

TABLE 10
Binding affinity of the humanized IL-25
Antibody Fabs to IL-25 proteins

| | KD (M) | kon(1/Ms) | kdis(1/s) |
|---|---|---|---|
| IL25Ab6 Fab | 1.86E−09 | 1.34E+05 | 2.49E−04 |
| IL25Ab7 Fab | 1.80E−09 | 1.61E+05 | 2.90E−04 |
| IL25Ab8 Fab | 1.78E−09 | 1.41E+05 | 2.51E−04 |
| IL25Ab3 Fab | 1.89E−09 | 1.50E+05 | 2.85E−04 |

### Example 9: Binding of Humanized Anti-IL-25 Antibodies to Human, Mouse and Cynomolgus IL-25 Recombinant Proteins

In this study, the ability of three humanized anti-IL-25 antibodies generated in hIgG1 (IL25Ab6 to Ab8) and hIgG1 YTE formats (IL25Ab9 to Ab11) binding to both human, mouse and cynomolgus IL-25 was evaluated using ELISA. Briefly, recombinant human IL-25 protein (Acro, catalog no: IL5-H4221, lot no: 401-20CNF1-UT), recombinant mouse IL-25 protein (Sino Biological Inc, catalog no: 50138-M07H, lot no: LC15AP0607) or recombinant cynomolgus IL-25 protein (Sino Biological Inc, catalog no: customized order, lot no: MB17MA2715) was directly coated to ELISA plates, respectively. Recombinant antibodies were then added to the plates followed by detection using goat-anti-human Kappa Light Chain antibody HRP (Novus, catalog no: NBP1-75064, Lot no: 68-188-010920). After the addition of ABTS (Moss Inc., catalog no: ABTS-1000, Lot no: 03086202) substrate, ELISA plates were read using an ELISA plate reader (Bioteck).

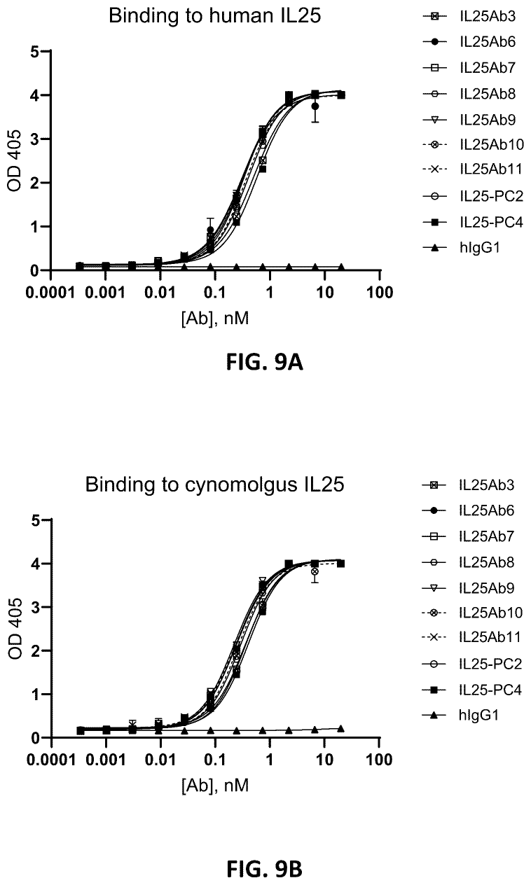

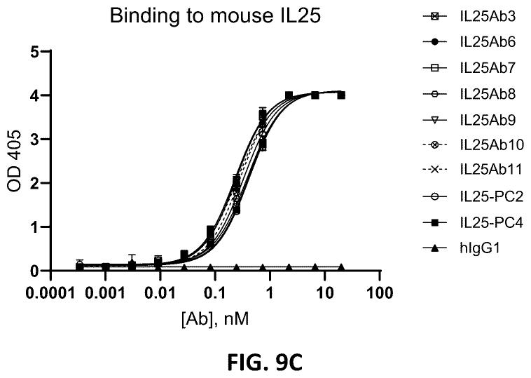

FIG. 9A shows that all humanized anti-IL-25 antibodies bound to the human IL-25 protein in a dose-dependent manner, with EC₅₀ values ranging from 0.302 nM to 0.404 nM. The mouse origin of anti-IL-25 specific antibody (IL25Ab3), the positive control antibodies IL-25-PC2 and IL-25-PC4 had EC50 values of 0.506 nM, 0.334 nM and 0.585 nM, respectively. The human IgG1 isotype control did not show any binding.

FIG. 9B shows that all humanized anti-IL-25 antibodies bound to the cynomolgus IL-25 protein in a dose-dependent manner, with EC50 values ranging from 0.222 nM to 0.295 nM. The mouse origin of anti-IL-25 specific antibody (IL25Ab3), the positive control antibodies IL-25-PC2 and IL-25-PC4 had EC50 values of 0.364 nM, 0.365 nM and 0.408 nM, respectively. The human IgG1 isotype control did not show any binding.

FIG. 9C shows that all humanized anti-IL-25 antibodies bound to the mouse IL-25 protein in a dose-dependent manner, with EC₅₀ values ranging from 0.232 nM to 0.332 nM. The mouse origin of anti-IL-25 specific antibody (IL25Ab3), the positive control antibodies IL-25-PC2 and IL-25-PC4 had EC50 values of 0.390 nM, 0.420 nM and 0.407 nM, respectively. The human IgG1 isotype control did not show any binding. These results are summarized in Table 11.

TABLE 11
Binding EC₅₀ of the IL-25 Antibodies to IL-25 proteins

| | Human IL-25 | Cynomolgus IL-25 | Mouse IL-25 ELISA |
|---|---|---|---|
| | ELISA EC₅₀, nM | ELISA EC₅₀, nM | EC₅₀, nM |
| IL25Ab3 | 0.506 | 0.364 | 0.390 |
| IL25Ab6 | 0.302 | 0.241 | 0.242 |
| IL25Ab7 | 0.327 | 0.245 | 0.243 |
| IL25Ab8 | 0.404 | 0.295 | 0.332 |
| IL25Ab9 | 0.312 | 0.222 | 0.232 |
| IL25Ab10 | 0.364 | 0.266 | 0.284 |
| IL25Ab11 | 0.318 | 0.239 | 0.258 |
| IL25-PC2 | 0.334 | 0.365 | 0.420 |
| IL25-PC4 | 0.585 | 0.408 | 0.407 |

### Example 10: Blocking of IL-25 Induced NF-kB Signaling

In this study, we examine the blocking activity of humanized anti-IL-25 antibodies using HEK-Blue™ IL-17 cells (InvivoGen, San Diego, CA) as described in the Example 4. In brief, the reporter cells were treated with recombinant human IL-25 at 5 ng/ml together with serially diluted IL-25 antibodies. After overnight incubation, the NF-κB response was determined using QUANTI-Blue™ Solution, a SEAP detection reagent, and reading the optical density (OD) at 655 nm.

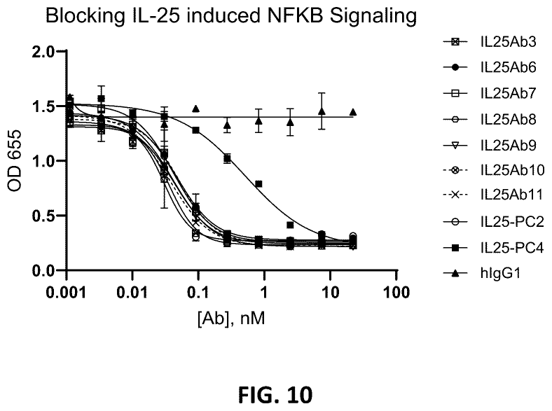

As shown in FIG. 10 and summarized in Table 12, all humanized anti-IL-25 antibodies effectively blocked IL-25 induced NF-kB signaling in a dose-dependent manner, with IC₅₀ values ranging from 0.037 nM to 0.052 nM. The mouse origin of anti-IL-25 specific antibody (IL25Ab3), the positive control antibodies IL-25-PC2 and IL-25-PC4 had IC₅₀ values of 0.037 nM, 0.030 nM and 0.513 nM, respectively. The human IgG1 isotype control did not show any blocking activity.

TABLE 12
Inhibitory activity of IL-25 Antibodiesin NFKB Signaling Assay

| | IC₅₀, nM |
|---|---|
| IL25Ab3 | 0.037 |
| IL25Ab6 | 0.048 |
| IL25Ab7 | 0.042 |
| IL25Ab8 | 0.041 |
| IL25Ab9 | 0.043 |
| IL25Ab10 | 0.052 |
| IL25Ab11 | 0.037 |
| IL25-PC2 | 0.030 |
| IL25-PC4 | 0.513 |

### Example 11: Blocking of IL-25 Induced CXCL1 Production

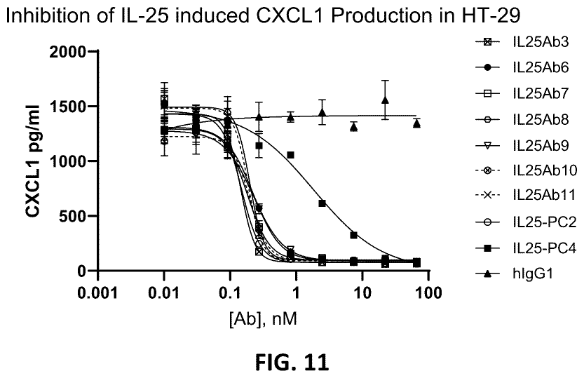

To determine whether the humanized anti-IL-25 antibodies can block the production of the inflammatory cytokine CXCL1 prompted by human IL-25, HT-29 cells endogenously expressed IL17 receptor were used as described in Example 5. In brief, HT-29 cells were stimulated with hIL-25 in a combination of serially diluted anti-IL-25 antibodies for 72 hours at 37° C. The supernatants were then collected for measurement of CXCL1 level using the Human CXCL1 ELISA Ready-SET-Go kit (R&D system #DY275). The results, which are presented in FIG. 11 and Table 13, demonstrate that the humanized anti-IL-25 antibodies retained strong blocking activity to IL-25 induced CXCL1 production, thus potentially reducing recruitment of immune cells and preventing the development of inflammation.

The IC₅₀ values of humanized antibodies (IL25Ab6-Ab11) ranged from 0.183 nM to 0.225 nM. The mouse origin of anti-IL-25 specific antibody (IL25Ab3), the positive control antibodies II-25-PC2 and IL-25-PC4 had IC₅₀ values of 0.144 nM, 0.148 nM and 1.84 nM, respectively. The human IgG1 isotype control did not show any blocking activity.

TABLE 13
Activity of IL-25 Antibodies in Blocking CXCL1 Production

| | IC50, nM |
|---|---|
| IL25Ab3 | 0.144 |
| IL25Ab6 | 0.225 |
| IL25Ab7 | 0.183 |
| IL25Ab8 | 0.199 |
| IL25Ab9 | 0.225 |
| IL25Ab10 | 0.198 |
| IL25Ab11 | 0.187 |
| IL25-PC2 | 0.148 |
| IL25-PC4 | 1.84 |

### Example 12: Blocking of IL-25 Induced IL-5 Production

To further characterize the biologic functions of humanized IL-25 antibodies and evaluate their potential in treating inflammatory disease, a human PBMC assay was performed as described in Example 6. In brief, IL-25 induced a significant amount of IL-5 production by the human PBMCs in the presence of 30 U/ml recombinant hIL-2. Blocking activity of humanized anti-IL-25 activities was determined to indicate their suppressive activity of the type 2 inflammatory response.

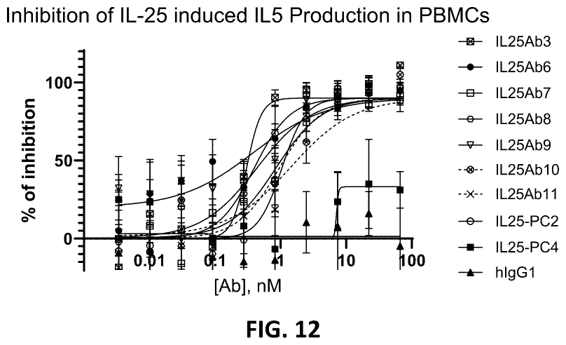

As shown in FIG. 12 and summarized in Table 14, all humanized IL-25 antibodies effectively blocked IL-25 induced IL-5 production in a dose-dependent manner, with IC₅₀ values ranging from 0.381 nM to 1.43 nM. The mouse origin of anti-IL-25 specific antibody (IL25Ab3), the positive control antibodies IL-25-PC2 and IL-25-PC4 had IC₅₀ values of 0.302 nM, 0.394 nM and 7.15 nM, respectively. The human IgG1 isotype control did not show any blocking activity.

TABLE 14
Activity of IL-25 Antibodies in Blocking IL-5 Production

| | IC50, nM |
|---|---|
| IL25Ab3 | 0.302 |
| IL25Ab6 | 0.381 |
| IL25Ab7 | 0.850 |
| IL25Ab8 | 1.04 |
| IL25Ab9 | 0.435 |
| IL25Ab10 | 1.43 |
| IL25Ab11 | 0.953 |
| IL25-PC2 | 0.394 |
| IL25-PC4 | 7.15 |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. An anti-IL-25 antibody comprising:
(a) a heavy chain variable region comprising the amino acid sequence of SEQ ID NO: 23 (HCDR1); the amino acid sequence of SEQ ID NO: 49 (HCDR2); and the amino acid sequence of SEQ ID NO: 25 (HCDR3); and a light chain variable region comprising the amino acid sequence of SEQ ID NO: 50 (LCDR1); the amino acid sequence of SEQ ID NO: 51 (LCDR2); and the amino acid sequence of SEQ ID NO: 28 (LCDR3);
(b) a heavy chain variable region comprising the amino acid sequence of SEQ ID NO: 23 (HCDR1); the amino acid sequence of SEQ ID NO: 52 (HCDR2); and the amino acid sequence of SEQ ID NO: 25 (HCDR3); and a light chain variable region comprising the amino acid sequence of SEQ ID NO: 50 (LCDR1); the amino acid sequence of SEQ ID NO: 51 (LCDR2); and the amino acid sequence of SEQ ID NO: 28 (LCDR3);
(c) a heavy chain variable region comprising the amino acid sequence of SEQ ID NO: 11 (HCDR1); the amino acid sequence of SEQ ID NO: 12 (HCDR2); and the amino acid sequence of SEQ ID NO: 13 (HCDR3); and a light chain variable region comprising the amino acid sequence of SEQ ID NO: 14 (LCDR1); the amino acid sequence of SEQ ID NO: 15 (LCDR2); and the amino acid sequence of SEQ ID NO: 16 (LCDR3);
(d) a heavy chain variable region comprising the amino acid sequence of SEQ ID NO: 17 (HCDR1); the amino acid sequence of SEQ ID NO: 18 (HCDR2); and the amino acid sequence of SEQ ID NO: 19 (HCDR3); and a light chain variable region comprising the amino acid sequence of SEQ ID NO: 20 (LCDR1); the amino acid sequence of SEQ ID NO: 21 (LCDR2); and the amino acid sequence of SEQ ID NO: 22 (LCDR3);
(e) a heavy chain variable region comprising the amino acid sequence of SEQ ID NO: 23 (HCDR1); the amino acid sequence of SEQ ID NO: 24 (HCDR2); and the amino acid sequence of SEQ ID NO: 25 (HCDR3); and a light chain variable region comprising the amino acid sequence of SEQ ID NO: 26 (LCDR1); the amino acid sequence of SEQ ID NO: 27 (LCDR2); and the amino acid sequence of SEQ ID NO: 28 (LCDR3);
(f) a heavy chain variable region comprising the amino acid sequence of SEQ ID NO: 29 (HCDR1); the amino acid sequence of SEQ ID NO: 30 (HCDR2); and the amino acid sequence of SEQ ID NO: 31 (HCDR3); and a light chain variable region comprising the amino acid sequence of SEQ ID NO: 32 (LCDR1); the amino acid sequence of SEQ ID NO: 33 (LCDR2); and the amino acid sequence of SEQ ID NO: 34 (LCDR3); or
(g) a heavy chain variable region comprising the amino acid sequence of SEQ ID NO: 35 (HCDR1); the amino acid sequence of SEQ ID NO: 36 (HCDR2); and the amino acid sequence of SEQ ID NO: 37 (HCDR3); and a light chain variable region comprising the amino acid sequence of SEQ ID NO: 38 (LCDR1); the amino acid sequence of SEQ ID NO: 39 (LCDR2); and the amino acid sequence of SEQ ID NO: 40 (LCDR3).

2. The anti-IL-25 antibody or an antigen-binding fragment thereof of claim 1, wherein the antibody or antigen-binding fragment thereof comprises a heavy chain variable region comprising the amino acid sequence of any one of SEQ ID NOs: 1, 3, 5, 7, 9, 44 or 47; and a light chain variable region comprising the amino acid sequence of any one of SEQ ID NOs: 2, 4, 6, 8, 10, 45, 46 or 48.

3. The anti-IL-25 antibody of claim 1, wherein the antibody comprises:
(a) a heavy chain variable region sequence of SEQ ID NO: 44 and a light chain variable region sequence of SEQ ID NO: 45;
(b) a heavy chain variable region sequence of SEQ ID NO: 44 and a light chain variable region sequence of SEQ ID NO: 46;
(c) a heavy chain variable region sequence of SEQ ID NO: 47_and a light chain variable region sequence of SEQ ID NO: 48;
(d) a heavy chain variable region sequence of SEQ ID NO: 1 and a light chain variable region sequence of SEQ ID NO: 2;
(e) a heavy chain variable region sequence of SEQ ID NO: 3 and a light chain variable region sequence of SEQ ID NO: 4;
(f) a heavy chain variable region sequence of SEQ ID NO: 5 and a light chain variable region sequence of SEQ ID NO: 6;
(g) a heavy chain variable region sequence of SEQ ID NO: 7 and a light chain variable region sequence of SEQ ID NO: 8; or
h) a heavy chain variable region sequence of SEQ ID NO: 9 and a light chain variable region sequence of SEQ ID NO: 10.

4. The anti-IL-25 antibody of claim 1, wherein the antibody is an anti-human anti-IL-25 antibody.

5. The anti-IL-25 antibody of claim 1, wherein the antibody is a full-length antibody comprising a human IgG1 constant region selected from SEQ ID NO: 55 or SEQ ID NO: 56.

6. The anti-IL-25 antibody of claim 1, wherein the antibody is an antibody fragment.

7. The anti-IL-25 antibody of claim 4, wherein the antibody fragment is selected from the group consisting of: Fab, Fab, F(ab)2, Fd, Fv, scFv and scFv-Fc fragment, a single-chain antibody, a minibody, and a diabody.

8. The anti-IL-25 antibody of claim 1, wherein the antibody is a monoclonal antibody.

9. The anti-IL-25 antibody of claim 1, wherein the antibody is a human antibody.

10. The anti-IL-25 antibody of claim 1, wherein the antibody is a murine antibody.

11. The anti-IL-25 antibody of claim 1, wherein the antibody is a chimeric antibody.

12. The anti-IL-25 antibody of claim 1, wherein the antibody is a bispecific or a multi-specific antibody.

13. The anti-IL-25 antibody of claim 1, wherein the antibody is a humanized antibody.

14. A pharmaceutical composition comprising the antibody of claim 1 and a pharmaceutically acceptable carrier.

15. A method of treating and/or preventing a Type 2 inflammation disease, autoimmune disease, allergic disorder, or cancer in a subject in need thereof, the method comprising: administering to the subject the antibody of claim 1.

16. A composition of polynucleotides comprising:
a) a first polynucleotide coding for a heavy chain variable region comprising the amino acid sequence set forth in SEQ ID NO: 1, 3, 5, 7, 9, 44, or 47; and
b) a second polynucleotide coding for a light chain variable region comprising the amino acid sequence set forth in SEQ ID NO: 2, 4, 6, 8, 10, 45, 46, or 48.

17. A composition of vectors comprising:
a) a first vector comprising the first polynucleotide of claim 16; and
b) a second vector comprising the second polynucleotide of claim 16.

18. A cell comprising the vector composition according to claim 17.

19. A method for the production of an anti-IL-25 antibody according to claim 1, the method comprising culturing the cell of claim 18 in a culture media; and recovering the anti-IL-25 antibody from the media.


SEQUENCE LISTING (57 sequences)

SEQ ID NO: 1 - protein, 123 aa, synthetic construct
  source 1..123: note = IL25Ab1
QVQLKESGPGLVAPSQSLSITCTVSGFSLTDYGINWVRQPPGKGLEWLGMIWGDGVTHYNSVLKSRLSISKDNSMSQIFLKMNSLQTDDTARYYCARTLFYDGTLYYYAMDYWGQGTSVTVSS

SEQ ID NO: 2 - protein, 108 aa, synthetic construct
  source 1..108: note = IL25Ab1
QIVLTQSPATMSASPGEKVTMTCSASSSVTYMYWYQQKTGSSPRLLIYDTPNLASGVPVRFSGSGSGTSYSLTISRVEAEDAATYYCQQWSSYPHTWTFGGGTKLEIK

SEQ ID NO: 3 - protein, 120 aa, synthetic construct
  source 1..120: note = IL25Ab2
QVTLKESGPGILQPSQTLSLTCSFSGFSLSTSGMGVGWIRQPSGKGLEWLAHIWWDDVKQYNPALQSRLTISKDTSSSQVFLKIASVDIADIATYYCARIGPVHHGFPYWGQGALVTVSS

SEQ ID NO: 4 - protein, 108 aa, synthetic construct
  source 1..108: note = IL25Ab2
QIVLTQSPAIMSAFPGEKVTMSCSASSSVNYMYWHQQKPGSSPRLLIYDTSNLASGVPVRFSGSGSGTSYSLTISRMEAEDAATYYCQQWTSYPPMYTFGGGTKLEIK

SEQ ID NO: 5 - protein, 125 aa, synthetic construct
  source 1..125: note = IL25Ab3
EVQLQQSGPELVKPGGSVRISCKASGYSFTGYTMNWVKQSHGKNLEWIGLINPDNGGTSHKQRFMGKATLTVDKSSSTAYMELLSLTSEDSAVYYCAREGYYGPSPFHHYAMDYWGQGTSVTVSS

SEQ ID NO: 6 - protein, 107 aa, synthetic construct
  source 1..107: note = IL25Ab3
DIQMTQTTSSLSASLGDRVTISCSASQDISNYLNWYQQKPDGTIKLLIYYTSTLHSGVPSRFSGSGSGTDYSLTISNLEPEDIATYYCQQYSKLPYTFGGGTKLEIK

SEQ ID NO: 7 - protein, 121 aa, synthetic construct
  source 1..121: note = IL25Ab4
QVTLKESGPGILQPSQTLSLTCSFSGFSLSTSGMGVSWIRQPSGKGLEWLAHIYWDDAKFYNPSLKSRVTISKDTSRNQVFLRITSVDTADIATYYCARRGYDNYGGFVDWGQGTLVTVSS

SEQ ID NO: 8 - protein, 108 aa, synthetic construct
  source 1..108: note = IL25Ab4
ENVLIQSPAIMSASLGEKVTMSCRASSSVNYMYWYQQKSDAPPKLWIYYTSNLAPGVPGRFSGSGSGNSYSLTISSMEGEDAATYYCQQFTSYPYMYTFGGGTKLEIK

SEQ ID NO: 9 - protein, 119 aa, synthetic construct
  source 1..119: note = IL25Ab5
QVQLQQWGAGLLKPSETLSLTCAVYGGSFSAYYWSWIRQSPGKGLEWIGEINHGGNTNYSPSLKSRVTISVDTSKSQFSLKLSSVTAADTAAYYCARGPGITSWAFDFWDQGTMVTVSS

SEQ ID NO: 10 - protein, 107 aa, synthetic construct
  source 1..107: note = IL25Ab5
DIQMTQSPSSLSASVGDRVTIACQASQDINNYLNWYQQKPGKAPKLLIYDASNLETGVPSRFSGNGSGTDFTFTISSLQPEDIATFYCQQYDTLPYTFGQGTKLEIR

SEQ ID NO: 11 - protein, 5 aa, synthetic construct
DYGIN

SEQ ID NO: 12 - protein, 16 aa, synthetic construct
MIWGDGVTHYNSVLKS

SEQ ID NO: 13 - protein, 15 aa, synthetic construct
TLFYDGTLYYYAMDY

SEQ ID NO: 14 - protein, 10 aa, synthetic construct
SASSSVTYMY

SEQ ID NO: 15 - protein, 7 aa, synthetic construct
DTPNLAS

SEQ ID NO: 16 - protein, 11 aa, synthetic construct
QQWSSYPHTWT

SEQ ID NO: 17 - protein, 7 aa, synthetic construct
TSGMGVG

SEQ ID NO: 18 - protein, 16 aa, synthetic construct
HIWWDDVKQYNPALQS

SEQ ID NO: 19 - protein, 10 aa, synthetic construct
IGPVHHGFPY

SEQ ID NO: 20 - protein, 10 aa, synthetic construct
SASSSVNYMY

SEQ ID NO: 21 - protein, 7 aa, synthetic construct
DTSNLAS

SEQ ID NO: 22 - protein, 11 aa, synthetic construct
QQWTSYPPMYT

SEQ ID NO: 23 - protein, 5 aa, synthetic construct
GYTMN

SEQ ID NO: 24 - protein, 17 aa, synthetic construct
LINPDNGGTSHKQRFMG

SEQ ID NO: 25 - protein, 16 aa, synthetic construct
EGYYGPSPFHHYAMDY

SEQ ID NO: 26 - protein, 11 aa, synthetic construct
SASQDISNYLN

SEQ ID NO: 27 - protein, 7 aa, synthetic construct
YTSTLHS

SEQ ID NO: 28 - protein, 9 aa, synthetic construct
QQYSKLPYT

SEQ ID NO: 29 - protein, 7 aa, synthetic construct
TSGMGVS

SEQ ID NO: 30 - protein, 16 aa, synthetic construct
HIYWDDAKFYNPSLKS

SEQ ID NO: 31 - protein, 11 aa, synthetic construct
RGYDNYGGFVD

SEQ ID NO: 32 - protein, 10 aa, synthetic construct
RASSSVNYMY

SEQ ID NO: 33 - protein, 7 aa, synthetic construct
YTSNLAP

SEQ ID NO: 34 - protein, 11 aa, synthetic construct
QQFTSYPYMYT

SEQ ID NO: 35 - protein, 5 aa, synthetic construct
AYYWS

SEQ ID NO: 36 - protein, 16 aa, synthetic construct
EINHGGNTNYSPSLKS

SEQ ID NO: 37 - protein, 11 aa, synthetic construct
GPGITSWAFDF

SEQ ID NO: 38 - protein, 11 aa, synthetic construct
QASQDINNYLN

SEQ ID NO: 39 - protein, 7 aa, synthetic construct
DASNLET

SEQ ID NO: 40 - protein, 9 aa, synthetic construct
QQYDTLPYT

SEQ ID NO: 41 - protein, 177 aa, Homo sapiens
  source 1..177: note = interleukin-25 isoform 1 precursor
MRERPRLGEDSSLISLFLQVVAFLAMVMGTHTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 42 - protein, 169 aa, Mus musculus
  source 1..169: note = NP_542767.1 interleukin-25 precursor
MYQAVAFLAMIVGTHTVSLRIQEGCSHLPSCCPSKEQEPPEEWLKWSSASVSPPEPLSHTHHAESCRASKDGPLNSRAISPWSYELDRDLNRVPQDLYHARCLCPHCVSLQTGSHMDPLGNSVPLYHNQTVFYRRPCHGEEGTHRRYCLERRLYRVSLACVCVRPRVMA

SEQ ID NO: 43 - protein, 176 aa, Macaca fascicularis
  source 1..176: note = XP_005560918.1 interleukin-25 isoform X1
MRGQPRLGENSSLISLFLQVIAFLAMVMGTHTYSHWPSCCPSKGYPSEELLRWSTVPVPPLKPASLDFHSVSCRASEDGPLNSRAISPWRYELDRDLNRLPQDLYHARCLCPHCVSLQTGSHMDPRGNSELLYHNQTVFYRRPCHGKKGNHKGYCLERRLYRVSLACVCVRPRVMG

SEQ ID NO: 44 - protein, 125 aa, synthetic construct
  source 1..125: note = IL25Ab6, Ab7, Ab9, Ab10_VH
QVQLVQSGAEVKKPGASVKVSCKASGYSFTGYTMNWVRQAPGQGLEWMGLINPDNGGTSYKQKFQGRVTMTVDKSISTAYMELSRLRSDDTAVYYCAREGYYGPSPFHHYAMDYWGQGTLVTVSS

SEQ ID NO: 45 - protein, 107 aa, synthetic construct
  source 1..107: note = IL25Ab6, Ab9_VL
DIQMTQSPSSLSASVGDRVTITCQASQDISNYLNWYQQKPGKAPKLLIYYTSTLHTGVPSRFSGSGSGTDYTFTISSLQPEDIATYYCQQYSKLPYTFGQGTKLEIK

SEQ ID NO: 46 - protein, 107 aa, synthetic construct
  source 1..107: note = IL25Ab7, Ab10_VL
DIQMTQSPSSLSASVGDRVTITCQASQDISNYLNWYQQKPDGTIKLLIYYTSTLHTGVPSRFSGSGSGTDYTFTISSLQPEDIATYYCQQYSKLPYTFGQGTKLEIK

SEQ ID NO: 47 - protein, 125 aa, synthetic construct
  source 1..125: note = IL25Ab8, Ab11_VH
QVQLVQSGAEVKKPGASVKVSCKASGYSFTGYTMNWVRQAHGKNLEWMGLINPDNGGTSHKQKFQGRVTMTVDKSISTAYMELSRLRSDDTAVYYCAREGYYGPSPFHHYAMDYWGQGTLVTVSS

SEQ ID NO: 48 - protein, 107 aa, synthetic construct
  source 1..107: note = IL25Ab8, Ab11_VL
DIQMTQSPSSLSASVGDRVTITCQASQDISNYLNWYQQKPDGTIKLLIYYTSTLHTGVPSRFSGSGSGTDYTLTISSLQPEDIATYYCQQYSKLPYTFGGGTKLEIK

SEQ ID NO: 49 - protein, 17 aa, synthetic construct
LINPDNGGTSYKQKFQG

SEQ ID NO: 50 - protein, 11 aa, synthetic construct
QASQDISNYLN

SEQ ID NO: 51 - protein, 7 aa, synthetic construct
YTSTLHT

SEQ ID NO: 52 - protein, 17 aa, synthetic construct
LINPDNGGTSHKQKFQG

SEQ ID NO: 53 - protein, 324 aa, Mus musculus
  source 1..324: note = Mouse IgG1 constant region
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 54 - protein, 107 aa, Mus musculus
  source 1..107: note = Mouse Kappa constant region
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 55 - protein, 330 aa, Homo sapiens
  source 1..330: note = Human IgG1 constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 330 aa, Homo sapiens
  source 1..330: note = Human IgG1 constant region with YTE mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 107 aa, Homo sapiens [sequence omitted]